(19) United States
(12) Patent Application Publication
(10) Pub. No.: US 2019/0192522 A1
(43) Pub. Date: Jun. 27, 2019

(54) INHIBITORS OF THE FIBROBLAST GROWTH FACTOR RECEPTOR 4 IN COMBINATION WITH CYCLIN-DEPENDENT KINASE INHIBITORS

(71) Applicant: BLUEPRINT MEDICINES CORPORATION, Cambridge, MA (US)

(72) Inventors: Margit HAGEL, Jamaica Plain, MA (US); Klaus HOEFLICH, Lexington, MA (US); Christoph LENGAUER, Cambridge, MA (US); Nicolas STRANSKY, Cambridge, MA (US); Christopher WINTER, Swampscott, MA (US); Lan XU, Wellesley, MA (US)

(73) Assignee: BLUEPRINT MEDICINES CORPORATION, Cambridge, MA (US)

(21) Appl. No.: 16/331,341

(22) PCT Filed: Sep. 8, 2017
(86) PCT No.: PCT/US2017/050782

§ 371 Date: Mar. 7, 2019

Related U.S. Application Data
(60) Provisional application No. 62/385,121, filed on Sep. 8, 2016.
(60) Provisional application No. 62/385,117, filed on Sep. 8, 2016.

(51) Int. Cl. A61K 31/519; A61K 31/517; A61P 35/04

(52) U.S. Cl. CPC A61K 31/519; A61P 35/04; A61K 31/517

(57) ABSTRACT

Described herein are selective inhibitors of FGFR4, pharmaceutical compositions including such compounds, and combinations with other therapeutic agents, such as CDK inhibitors (e.g., CDK4/6 inhibitors), and methods of using such combinations.

### CLAIM OF PRIORITY

This application claims priority from U.S. Provisional Application No. 62/385,121, filed Sep. 8, 2016, and U.S. Provisional Application No. 62/385,117, filed Sep. 8, 2016, each of which is incorporated by reference herein in its entirety.

### BACKGROUND

Fibroblast growth factor receptor 4 (FGFR4) is a protein that in humans is encoded by the FGFR4 gene. This protein is a member of the fibroblast growth factor receptor family, where amino acid sequence was highly conserved between members throughout evolution. FGFR family members 1-4 differ from one another in their ligand affinities and tissue distribution. A full-length representative protein consists of an extracellular region composed of three immunoglobulin-like domains, a single hydrophobic membrane-spanning segment, and a cytoplasmic tyrosine kinase domain. The extracellular portion of the protein interacts with fibroblast growth factors, setting in motion a cascade of downstream signals, ultimately influencing mitogenesis and differentiation. The genomic organization of the FGFR4 gene encompasses eighteen exons. Although alternative splicing has been observed, there is no evidence that the C-terminal half of the IgIII domain of this protein varies between three alternate forms, as indicated for FGFR1-3.

To date, there are no approved potent and selective FGFR4 inhibitors. While several FGFR inhibitors are currently in clinical trials to treat cancers with FGFR1-3 aberrations, many of these inhibitors exhibit promiscuous kinome activity or moderate to weak potency against FGFR4. Lack of kinome selectivity can result in toxicity due to off-target effects. Specifically, on-target, dose-limiting toxicities have been observed in both animals and patients administered FGFR1 and 3 inhibitors (Dieci, M V et al. (2013), Cancer Discov., 3:264-79). For example, ectopic mineralization, characterized by inappropriate calcium-phosphorus deposition in soft tissue, has been observed in rats treated with an FGFR1 inhibitor (Brown, A P et al. (2005), Toxicol. Pathol., p. 449-455). Inhibition of FGFR1 and 3 can also lead to hyperphosphatemia. This suggests that selective inhibition of FGFR4 without inhibition of other isoforms of FGFR, including FGFR1 and FGFR3, may be desirable in order to avoid certain toxicities. FGFR4 preferentially binds fibroblast growth factor 19 (FGF19) and has recently been associated with the progression of certain sarcomas, renal cell cancer, breast cancer, and liver cancer. For instance, aberrant signaling through the fibroblast growth factor 19 (FGF19)/FGFR4 signaling complex has been shown to cause hepatocellular carcinoma (HCC) in mice and has been implicated to play a similar role in humans.

Additionally, in many human cancers, cyclin dependent kinases (CDK) promote cancer cell growth. CDK inhibitors, such as cyclin dependent kinase 4/6 (CDK4/6) inhibitors, may be used to reduce cancer cell proliferation mediated at least in part by activated CDK pathways (e.g., an activated CDK4/6 pathway). A CDK4/6 inhibitor, 6-acetyl-8-cyclopentyl-5-methyl-2-{[5-(1-piperazinyl)-2-pyridinyl]amino}pyrido[2,3-d]pyrimidin-7(8H)-one (also referred to as palbociclib or PD0332991), was approved by the United States Food and Drug Administration in February 2015 to treat estrogen receptor (ER)-positive, human epidermal growth factor receptor 2 (HER2)-negative metastatic breast cancer in postmenopausal women. Chromosomal gains in HCC have been shown to result in focal amplification of FGF19 and CCND1 (Chiang, DY et al. (2008), Cancer Res. 68(16); 6779-88).

### BRIEF DESCRIPTION OF THE DRAWINGS

FIG. 1 depicts a table showing synergistic growth inhibition in cells for the combination of Compound 1 and palbociclib. The cells showed a dose dependent reduction in proliferation based on BrdU incorporation, with a synergy score of 5.47.

FIG. 2 depicts a heat map showing synergistic growth inhibition in cells for the combination of Compound 1 and palbociclib. The last heat map shows only 0.24% of the cells in the S-phase i.e., no division of cells.

FIG. 3 depicts a gel showing synergistic growth inhibition in cells treated with the combination of Compound 1 and palbociclib. Cell cycle analysis shows the majority of cells are trapped in G1, the first phase of the cell cycle, following inhibition with the combination.

FIG. 4 depicts a line graph showing the percent body weight change in Balb/c nude xenograft mice treated with Compound 1, palbociclib, and the combination of Compound 1 and palbociclib.

FIG. 5 depicts a line graph showing synergistic growth inhibition in vivo in Balb/c nude xenograft mice treated with the combination of Compound 1 and palbociclib in comparison to single agent Compound 1 and palbociclib.

FIG. 6 depicts end of study photomicrographs showing H&E staining of xenograft tumors from mice treated with vehicle (A), palbociclib (B), Compound 1 (C), or the combination of Compound 1 and palbociclib (D).

FIG. 7 depicts end of study photomicrographs showing Ki67 staining of xenograft tumors from mice treated with vehicle (A), palbociclib (B), Compound 1 (C), or the combination of Compound 1 and palbociclib (D).

FIG. 8 is a bar graph showing inhibition of the expression of proliferation markers Ki67 and phospho-Histone H3 for Balb/c nude mice treated with vehicle, Compound 1 (100 mg/kg), palbociclib (90 mg/kg), and the combination of Compound 1 and palbociclib.

FIG. 9 depicts a bar graph showing the results of the synergistic growth inhibition shown in vivo in Balb/c nude xenografts treated with combinations of Compound 1 and palbociclib.

FIGS. 10A and 10B depict line graphs showing in vivo activity of Compound 1 monotherapy in hepatocellular carcinoma (HCC) mouse models that are dependent on FGFR4 signaling.

### SUMMARY OF THE DISCLOSURE

In one aspect, the disclosure provides a method for treating a cancer (e.g., hepatocellular carcinoma or fibrolamellar hepatocellular carcinoma) in a subject. The method comprises administering a therapeutically effective amount of at least one FGFR4 inhibitor, e.g., at least one FGFR4 inhibitor described herein, in combination with at least one cyclin-dependent kinase (CDK) inhibitor described herein (e.g., at least one CDK4/6 inhibitor described herein).

In some embodiments, the cancer is hepatocellular carcinoma (HCC), breast cancer, ovarian cancer, lung cancer, liver cancer, a sarcoma, intrahepatic cholangiocarcinoma (ICC), esophagus cancer, large intestine cancer, colon cancer, head and neck cancer, or hyperlipidemia. In some embodiments, the cancer is hepatocellular carcinoma. In some embodiments, the hepatocellular carcinoma is unresectable. In some embodiments, the hepatocellular carcinoma is metastatic. In some embodiments, the cancer is fibrolamellar hepatocellular carcinoma. In some embodiments, the cancer is estrogen receptor (ER)-positive, human epidermal growth factor receptor 2 (HER2)-negative metastatic breast cancer.

In some embodiments, the cancer is pancreatic cancer (e.g., well or moderately differentiated metastatic pancreatic neuroendocrine tumors (pNET)), leukemia (e.g., acute myeloid leukemia or acute lymphoblastic leukemia), oligoastrocytoma, oligodendroglioma, liposarcoma, urothelial cancer, non-small cell lung cancer, squamous cell lung cancer, glioblastoma, thymic cancer, prostate cancer, esophagus cancer, large intestine cancer, colon cancer, head and neck cancer, or chordoma. In some embodiments, the cancer is advanced.

In some embodiments, the cancer is characterized by progressive brain metastases or recurrent, progressive, or refractory central nervous system tumors.

In some embodiments, the cancer is mediated by FGFR4.

In some embodiments, the cancer is characterized by an aberrant FGFR4 signaling pathway.

In some embodiments, the cancer is not treatable by palbociclib alone. For example, in some embodiments, the cancer is characterized by a mutated retinoblastoma protein.

In some embodiments, the cancer is characterized by overexpression of FGFR4, e.g., as compared to a reference standard (normal tissue).

In some embodiments, the cancer is characterized by amplified FGF19, e.g., as compared to a reference standard (normal tissue). For example, the FGF19 gene copy number (CN) in cancer cells is elevated (≥5 copies, ≥6 copies, ≥7 copies, ≥8 copies, ≥9 copies, ≥10 copies, ≥11 copies, ≥12 copies, ≥13 copies, ≥14 copies, ≥15 copies , ≥16 copies, ≥17 copies, ≥18 copies or more) compared to healthy/normal cells (with 2 copies or less). In some embodiments, the FGF19 gene copy number in liver cancer cells is elevated compared to healthy/normal liver cells. In some embodiments, the cancer is further characterized by having an intact FGFR4 signaling pathway (FGFR4, FGF19, and KLB). In some embodiments, analysis using nanostring technology or RNA sequencing is used to determine the presence of an intact FGFR4 signaling pathway in cell line model or a patient. Some examples of cell line models with an intact signaling pathway are Huh-7, JHH-7, and Hep 3B. Some examples of cell line models without an intact signaling pathway include PLC/PRF/5, SNU-182, SK-Hepl, SNU-387, SNU-423, and SNU-398. In some embodiments, the cell line model has very low expression of KLB in comparison to other members of the pathway (SNU-878). In some embodiments, the cancer is further characterized by wild-type retinoblastoma protein (R^{B}) and wild-type klotho beta.

In some embodiments, the cancer is characterized by amplified FGF19 and an intact G1 checkpoint i.e., R^{B} is wild type (not mutated) and CDK4 and CDK6 are wild-type (not mutated). In some embodiments, the cancer is characterized by amplified FGF19 and R^{B} status does not matter e.g., the R^{B} gene or protein may or may not be mutated.

In some embodiments, the cancer is characterized by aberrant FGF19 expression. In some embodiments, the cancer is characterized by overexpression of FGF19, e.g., as compared to a reference standard (e.g., normal tissue). For example, in cells that normally do not express FGF19, expression of FGF19 in cancer cells constitutes overexpression of FGF19 relative to a reference standard. In some embodiments, in healthy liver cells that normally do not express FGF19 (<1%), any expression of FGF19 >1% in liver cancer cells constitutes overexpression of FGF19 relative to healthy liver cells. In some embodiments, the expression of FGF19 is ≥1% (IHC positive). In some embodiments, the expression of FGF19 is <1% (IHC negative).

In some embodiments, the cancer is characterized by amplified FGF19 and overexpression of FGF19. In some embodiments, the cancer is further characterized by wild-type retinoblastoma protein and wild-type klotho beta.

In some embodiments, the cancer is characterized by FGF19 overexpression without statistically significant FGR19 amplification i.e., the FGF19 gene copy number is not elevated (below 5 copies) compared to a reference standard (normal tissue with two copies). In some embodiments, the cancer is further characterized by wild-type retinoblastoma protein and wild-type klotho beta.

In some embodiments, the cancer is characterized by wild-type retinoblastoma protein and wild-type klotho beta without statistically significant FGR19 overexpression or statistically significant FGR19 amplification.

In some embodiments, the at least one FGFR4 inhibitor is chosen from compounds of Formula (I) and pharmaceutically acceptable salts thereof, wherein:

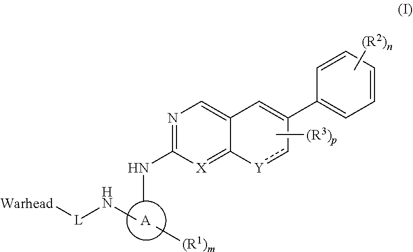

Warhead is a moiety capable of forming a covalent bond with a nucleophile;

dashed line is absent or a single bond;

ring A is a 3-8 membered aryl, heteroaryl, heterocyclic, or alicyclic group;

X is CH or N;

Y is CH or N—R⁴, wherein R⁴ is H or C₁₋₆ alkyl;

L is —[C(R⁵)(R⁶)]_{q}—, wherein each of R⁵ and R⁶ is independently H or C₁₋₆ alkyl, and wherein q is 0-4;

each of R¹-R³ is independently halo, cyano, optionally substituted C₁₋₆ alkoxy, hydroxy, oxo, amino, amido, alkyl urea, optionally substituted C₁₋₆ alkyl, or optionally substituted C₁₋₆ heterocyclyl;

m is 0-3;

n is 0-4; and

p is 0-2.

In some embodiments, ring A is phenyl (e.g., a 1,2-disubstituted phenyl); each of R² is independently halo or methoxy; n is 2 or 4; X is N; R¹ is methyl; or m is 1.

In some embodiments, the at least one FGFR4 inhibitor is chosen from compounds of Formula (II) and pharmaceutically acceptable salts thereof, wherein:

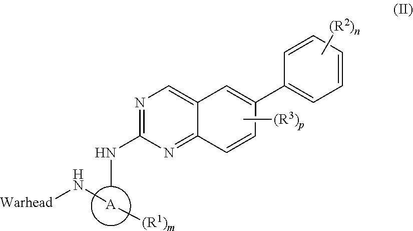

Warhead is a moiety capable of forming a covalent bond with a nucleophile;

ring A is a 3-8 membered monocyclic or bicyclic cycloalkyl or heterocyclyl group;

each of R¹ and R² is independently halo, cyano, C₁₋₆ alkoxy, hydroxy, oxo, amino, amido, sulfonyl, sulfonamido, ester, alkyl urea, C₁₋₆ alkyl, —C(O)O—, —C(O)—C₁₋₆ alkyl, —C(O)—C₁₋₆ alkylamino, C₁₋₆ heteroalkyl, heterocyclyl, or heterocyclylalkyl, wherein each of C₁₋₆ alkoxy, amino, amido, sulfonamido, ester, alkyl urea, C₁₋₆ alkyl, C₁₋₆ heteroalkyl, heterocyclyl, or heterocyclylalkyl is independently substituted with 0-5 occurrences of R⁴;

each R³ is independently halo;

each R⁴ is independently chosen from C₁₋₆ alkyl, C₁₋₆ alkoxy, halo, hydroxy, oxo, amino, cyano, cycloalkyl, and heterocyclyl;

m is 0-3;

n is 0-4; and

p is 0-2.

In some embodiments embodiment, ring A is a 3-8 membered monocyclic cycloalkyl. In some embodiments, ring A is cyclobutyl, cyclopentyl, or cyclohexyl.

In some embodiments, ring A is a 3-8 membered bicyclic cycloalkyl.

In some embodiments, ring A is a 3-8 membered heterocyclyl. In some embodiments, ring A is pyrrolidinyl, piperidinyl, tetrahydrofuranyl, or tetrahydropyranyl.

In some embodiment, the at least one FGFR4 inhibitor is chosen from compounds of Formula (III) and pharmaceutically acceptable salts thereof, wherein:

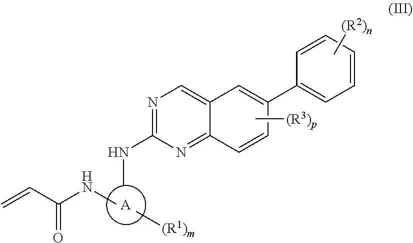

ring A is a 3-6 membered cycloalkyl or heterocyclyl;

each R¹ is independently halo, cyano, C₁₋₆ alkoxy, hydroxy, oxo, amino, amido, sulfonyl, sulfonamido, ester, alkyl urea, C₁₋₆ alkyl, —C(O)O—, —C(O)—C₁₋₆ alkyl, —C(O)—C₁₋₆ alkylamino, or C₁₋₆ heteroalkyl;

each R² is independently halo or C₁₋₆ alkoxy;

each R³ is independently halo; and

m is 0-1;

n is 0-4; and

p is 0-1.

In some embodiments, ring A is a 3-6 membered cycloalkyl.

In some embodiments, ring A is a 3-6 membered heterocyclyl.

In some embodiments, ring A is cyclobutyl, cyclopentyl, cyclohexyl, pyrrolidinyl, piperidinyl, tetrahydrofuranyl, or tetrahydropyranyl.

In the compounds disclosed herein, a warhead is a moiety that is reactive with a nucleophile, for example, capable of forming a covalent bond with a nucleophile. Examples of warheads include, without limitation, those disclosed in, for example, U.S. Pat. No. 9,434,700, which is incorporated herein by reference in its entirety. For example, warheads include, without limitation, alkyl halides, alkyl sulfonates, heteroaryl halides, epoxides, haloacetamides, maleimides, sulfonate esters, alpha-beta unsaturated ketones, alpha-beta unsaturated esters, vinyl sulfones, propargyl amides, and acrylamides. In some warheads, such as acrylamides and propargyl amides, the nitrogen of the warhead is the adjacent nitrogen in the formulae shown above.

Non-limiting examples of warheads include:

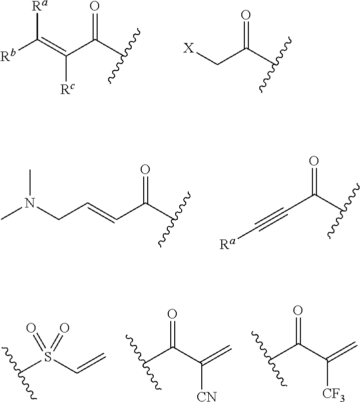

wherein X is a leaving group (e.g., halo) or an activated hydroxyl moiety (e.g., triflate); and

each of R^{a}, R^{b}, and R^{c} is, independently, H, substituted or unsubstituted C₁₋₄ alkyl, substituted or unsubstituted C₃₋₄ cycloalkyl, or cyano.

In the formulae shown above, the warheads are typically attached to a nitrogen atom on the inhibitor. In other embodiments, the warhead can alternatively be attached to an atom other than nitrogen. Additional non-limiting examples of warheads include:

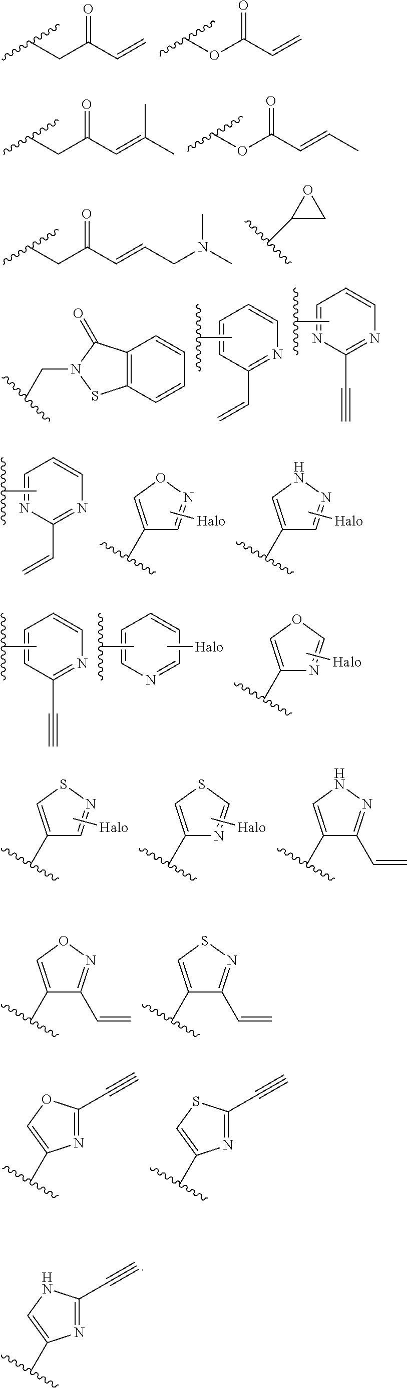

Other examples of warheads can be found, e.g., in WO 2010/028236 and WO 2011/034907, each of which is incorporated by reference herein in its entirety.

In some embodiments, the at least one FGFR4 inhibitor is chosen from selective FGFR4 inhibitors.

In some embodiments, the at least one FGFR4 inhibitor is chosen from selective covalent FGFR4 inhibitors. In some embodiments, the selective covalent FGFR inhibitor covalently binds to Cys552 of FGFR4.

In some embodiments, the at least one FGFR4 inhibitor is chosen from compounds and pharmaceutically acceptable salts thereof as disclosed in U.S. Pat. Nos. 8,802,697, 9,266,883, 9,321,786, 9,745,311, WO 2017/070708, and U.S. Pat. No. 9,533,988, WO 2014/011900, WO 2015/061572, WO 2015/108992, WO 2010/026291, WO 2011/135376, WO 2011/016528, WO 2015/057963, WO2015/057938, WO2016/064960, WO 2016/134294, WO 2016/134314, WO 2016/134320, US 2016/0115164 each of which is incorporated herein by reference in its entirety.

In some embodiments, the at least one FGFR4 inhibitor is chosen from N-((3S,4S)-3-((6-(2,6-dichloro-3,5-dimethoxyphenyl)quinazolin-2-yl)amino)tetrahydro-2H-pyran-4-yl)acrylamide (Compound 1):

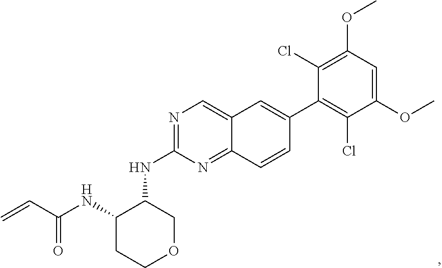

N-(2-((6-(2,6-dichloro-3,5-dimethoxyphenyl)quinazolin-2-yl)amino)-3-methylphenyl)acrylamide (Compound 2):

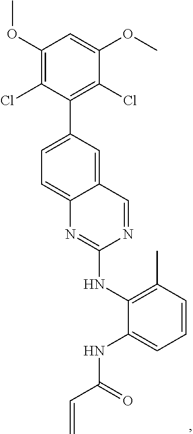 [SMILES: C=CC(=O)Nc1cccc(C)c1Nc1ncc2cc(-c3c(Cl)c(OC)cc(OC)c3Cl)ccc2n1]

and pharmaceutically acceptable salts thereof.

In some embodiments, the at least one FGFR4 inhibitor is chosen from N-[2-[[6-[(2,6-dichloro-3,5-dimethoxyphenyl)carbamoyl-methylamino]pyrimidin-4-yl]amino]-5-(4-ethylpiperazin-1-yl)phenyl]prop-2-enamide (also referred to as H3B-6527), N-[5-cyano-4-(2-methoxyethylamino)pyridin-2-yl]-7-formyl-6-[(4-methyl-2-oxopiperazin-1-yl)methyl]-3,4-dihydro-2H-1,8-naphthyridine-1-carboxamide (also referred to as FGF401), 3-(2,6-dichloro-3,5-dimethoxyphenyl)-1-(6-(4-(4-ethylpiperazin-1-yl)-phenylamino)pyrimidin-4-yl)-1-methylurea (also referred to as infigratinib or BGJ398, 2-(4-(2-(5-(1-(3,5-dichloropyridin-4-yl)ethoxy)-1H-indazol-3yl)vinyl)-1H-pyrazol-1-yl)ethanol (also referred to as LY2874455), 1,2-Ethanediamine, N1-(3,5-dimethoxyphenyl)-N2-(1-methylethyl)-N1-[3-(1-methyl-1H-pyrazol-4-yl)-6-quinoxalinyl] (also referred to as erdafitinib), (5-amino-1-(2-methyl-3H-benzo[d]imidazol-5-yl)-1H-pyrazol-4-yl)(1H-indol-2-yl)methanone (also referred to as CH5183284 (Debio-1347)) and pharmaceutically acceptable salts thereof. In some embodiments, the at least one FGFR4 inhibitor is an FGFR4 monoclonal antibody (e.g., U3-1784).

In some embodiments, the at least one CDK inhibitor (e.g., the at least one CDK4/6 inhibitor) is chosen from compounds and pharmaceutically acceptable salts thereof as disclosed in U.S. Pat. No. 6,936,612, U.S. Patent Application Publication No. 2013/0035336, U.S. Patent Application Publication No. 2013/0150342, U.S. Patent Application Publication No. 2016/0002223, WO 2011/101409, and WO 2014/128588, each of which is incorporated herein by reference in its entirety.

In some embodiments, the at least one CDK inhibitor (e.g., the at least one CDK4/6 inhibitor) is chosen from 6-acetyl-8-cyclopentyl-5-methyl-2-{[5-(1-piperazinyl)-2-pyridinyl]amino}pyrido[2,3-d]pyrimidin-7(8H)-one (also referred to as palbociclib or PD0332991) and pharmaceutically acceptable salts thereof.

In some embodiments, 125 mg of palbociclib or an equivalent amount of a pharmaceutically acceptable salt of palbociclib is administered once daily. In some embodiments, less than 125 mg of palbociclib or an equivalent amount of a pharmaceutically acceptable salt of palbociclib is administered once daily. In some embodiments, palbociclib or a pharmaceutically acceptable salt thereof is taken with food. In some embodiments, palbociclib is administered in combination with letrozole 2.5 mg once daily.

In some embodiments, the at least one CDK inhibitor (e.g., the at least one CDK4/6 inhibitor) is chosen from: 7-cyclopentyl-N,N-dimethyl-2-((5-(piperazin-1-yl)pyridin-2-yl)amino)-7H-pyrrolo[2,3-d]pyrimidine-6-carboxamide (also referred to as LEE011); 2-(2-chlorophenyl)-5,7-dihydroxy-8-[(3 S,4R)-3-hydroxy-1-methyl-4-piperidinyl]-4-chromenone (also referred to as flavopiridol, HMR-1275, or alvocidib); N-(5-((4-ethylpiperazin-1-yl)methyl)pyridin-2-yl)-5-fluoro-4-(4-fluoro-1-isopropyl-2-methyl-1H-benzo[d]imidazol-6-yl)pyrimidin-2-amine (also referred to as LY2835219 or abemaciclib); GZ38-1; and pharmaceutically acceptable salts thereof.

In some embodiments, the at least one CDK inhibitor is chosen from abemaciclib, flavopiridol, ribociclib, and pharmaceutically acceptable salts thereof.

The compounds of the present disclosure inhibit FGFR4 and/or CDK4/6, and therefore the present combination may be capable of treating diseases wherein the underlying pathology is (at least in part) mediated by activated CDK4/6 and/or FGFR4 pathway. Such diseases include cancer and other diseases in which there is a disorder of cell proliferation, apoptosis, or differentiation. In one aspect, the disclosure provides a method for treating a cancer (e.g., hepatocellular carcinoma) comprising administering to a subject a therapeutically effective amount of N-((3S,4S)-3-((6-(2,6-dichloro-3,5-dimethoxyphenyl)quinazolin-2-yl)amino)tetrahydro-2H-pyran-4-yl)acrylamide (Compound 1) or a pharmaceutically acceptable salt thereof in combination with 6-acetyl-8-cyclopentyl-5-methyl-2-{[5-(1-piperazinyl)-2-pyridinyl]amino}pyrido[2,3-d]pyrimidin-7(8H)-one (also referred to as palbociclib or PD0332991) or a pharmaceutically acceptable salt thereof.

In one aspect, the disclosure provides a method for treating a cancer (e.g., hepatocellular carcinoma) comprising administering to a subject a therapeutically effective amount of N-((3S,4S)-3-((6-(2,6-dichloro-3,5-dimethoxyphenyl)quinazolin-2-yl)amino)tetrahydro-2H-pyran-4-yl)acrylamide (Compound 1) or a pharmaceutically acceptable salt thereof in combination with 6-acetyl-8-cyclopentyl-5-methyl-2-{[5-(1-piperazinyl)-2-pyridinyl]amino}pyrido[2,3-d]pyrimidin-7(8H)-one (also referred to as palbociclib or PD0332991) or a pharmaceutically acceptable salt thereof, wherein the cancer is characterized by overexpression of FGF19.

In one aspect, the disclosure provides a method for treating a cancer (e.g., hepatocellular carcinoma) comprising administering to a subject a therapeutically effective amount of N-((3S,4S)-3-((6-(2,6-dichloro-3,5-dimethoxyphenyl)quinazolin-2-yl)amino)tetrahydro-2H-pyran-4-yl)acrylamide (Compound 1) or a pharmaceutically acceptable salt thereof in combination with 6-acetyl-8-cyclopentyl-5-methyl-2-{[5-(1-piperazinyl)-2-pyridinyl]amino}pyrido[2,3-d]pyrimidin-7(8H)-one (also referred to as palbociclib or PD0332991) or a pharmaceutically acceptable salt thereof, wherein the cancer is characterized by amplified FGF19.

In some embodiments, the cancer is hepatocellular carcinoma, breast cancer, ovarian cancer, lung cancer, liver cancer, a sarcoma, esophagus cancer, large intestine cancer, colon cancer, head and neck cancer, or hyperlipidemia. In some embodiments, the cancer is hepatocellular carcinoma. In some embodiments, the cancer is fibrolamellar hepatocellular carcinoma. In some embodiments, the cancer is fibrolamellar hepatocellular carcinoma. In some embodiments, the cancer is estrogen receptor (ER)-positive, human epidermal growth factor receptor 2 (HER2)-negative metastatic breast cancer.

In some embodiments, the cancer is pancreatic cancer (e.g., well or moderately differentiated metastatic pancreatic neuroendocrine tumors (pNET)), leukemia (e.g., acute myeloid leukemia or acute lymphoblastic leukemia), oligoastrocytoma, oligodendroglioma, liposarcoma, urothelial cancer, non-small cell lung cancer, squamous cell lung cancer, glioblastoma, thymic cancer, prostate cancer, esophagus cancer, large intestine cancer, colon cancer, head and neck cancer, or chordoma. In some embodiments, the cancer is advanced. In some embodiments, the cancer is unresectable. In some embodiments, the cancer is metastatic. In some embodiments, the cancer is refractory.

In some embodiments, the cancer is characterized by progressive brain metastases or recurrent, progressive, or refractory central nervous system tumors.

In some embodiments, the cancer is esophagus cancer. In some embodiments, the cancer is large intestine cancer. In some embodiments, the cancer is colon cancer. In some embodiments, the cancer is head and neck cancer.

In some embodiments, Compound 1 or a pharmaceutically acceptable salt thereof is administered once or twice daily.

In some embodiments, Compound 1 or a pharmaceutically acceptable salt thereof is administered once daily. In some embodiments, up to 600 mg of Compound or an equivalent amount of a pharmaceutically acceptable salt of Compound 1 is administered once daily. For example, in some embodiments, 140 mg, 280 mg, 420 mg or 600 mg of Compound 1 or an equivalent amount of a pharmaceutically acceptable salt of Compound 1 is administered once daily. In some embodiments, Compund 1 or a pharmaceutically acceptable salt thereof is administered in the form of a tablet.

In some embodiments, 100 mg to 300 mg of Compound 1 or an equivalent amount of a pharmaceutically acceptable salt of Compound 1 is administered twice daily. For example, in some embodiments, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg of Compound 1 or an equivalent amount of a pharmaceutically acceptable salt of Compound 1 is administered twice daily. In some embodiments, 100 mg, 150 mg, 200 mg, or 300 mg of Compound 1 or an equivalent amount of a pharmaceutically acceptable salt of Compound 1 is administered twice daily. In some embodiments, Compound 1 or a pharmaceutically acceptable salt thereof is administered in the form of a tablet.

In some embodiments, the total daily dose of Compound 1 or an equivalent amount of a pharmaceutically acceptable salt of Compound 1 is less than 600 mg. In some embodiments, the total daily dose of Compound 1 or an equivalent amount of a pharmaceutically acceptable salt of Compound 1 is 200, 300, or 400 mg. In some embodiments, the time between administrations is ten to fourteen hours. In some embodiments, the time between administrations is at least eight hours.

In some embodiments, Compound 1 or a pharmaceutically acceptable salt thereof is administered once in the morning and once in the evening.

In some embodiments, 50 to 150 mg of palbociclib or an equivalent amount of a pharmaceutically acceptable salt of palbociclib is orally administered to the patient once daily. In some embodiments, 125 mg of palbociclib or an equivalent amount of a pharmaceutically acceptable salt of palbociclib is orally administered to the patient once daily. In some embodiments, less than 125 mg of palbociclib or an equivalent amount of a pharmaceutically acceptable salt of palbociclib is orally administered to the patient once daily.

In some embodiments, palbociclib is taken with food, optionally in combination with letrozole 2.5 mg once daily. In some embodiments, palbociclib is administered to the patient for twenty-one consecutive days, followed by seven days in which no palbociclib is administered to the patient. In some embodiments, the twenty-eight day administration schedule is repeated one or more times.

In some embodiments, 125 mg of palbociclib is administered once daily and 100 mg, 150 mg, 200 mg, or 300 mg of Compound 1 or an equivalent amount of a pharmaceutically acceptable salt of Compound 1 is administered twice daily.

In some embodiments, less than 125 mg of palbociclib is administered once daily and 100 mg, 150 mg, 200 mg, or 300 mg of Compound 1 or an equivalent amount of a pharmaceutically acceptable salt of Compound 1 is administered twice daily.

In some embodiments, the patient has been previously treated with a tyrosine kinase inhibitor e.g., sorafenib.

In some embodiments, the patient has not been previously treated with a tyrosine kinase inhibitor e.g., sorafenib.

In some embodiments, the cancer is mediated by FGFR4.

In some embodiments, the cancer is characterized by an aberrant FGFR4 signaling pathway.

Palbociclib sensitvity is largely dependent on retinoblastoma protein status. In some embodiments, the cancer is characterized by overexpression of FGF19 and wild type retinoblastoma protein. In some embodiments, the cancer is not treatable by palbociclib alone. For example, in some embodiments, the cancer is characterized by a mutated retinoblastoma protein. In some embodiments, the cancer is characterized by overexpression of FGF19 and mutated retinoblastoma protein. In some embodiments, the cancer is characterized by overexpression of FGF19, mutated retinoblastoma protein, and CCND1 amplification.

In some embodiments, the cancer is characterized by overexpression of FGFR4, e.g., as compared to a reference standard (e.g., normal tissue).

In some embodiments, the cancer is characterized by amplified FGF19, e.g., as compared to a reference standard (e.g., normal tissue). In some embodiments, the cancer is further characterized by wild-type retinoblastoma protein and wild-type klotho beta.

In some embodiments, the cancer is characterized by amplified FGF19 and an intact G1 checkpoint.

In some embodiments, the cancer is characterized by overexpression of FGF19, e.g., as compared to a reference standard. For example, in cells that normally do not express FGF19, expression of FGF19 in cancer cells constitutes overexpression of FGF19 relative to a reference standard. In some embodiments, the cancer is characterized by overexpression of FGF19 (>1%), no detectable FGF19 amplification, wild type FGFR4, wild type R^{B}, and wild type klotho beta. In some embodiments, the cancer is characterized by amplified FGF19 and overexpression of FGF19. In some embodiments, the cancer is further characterized by wild-type retinoblastoma protein and wild-type klotho beta.

In some embodiments, the cancer is characterized by FGR19 overexpression without statistically significant FGR19 amplification. In some embodiments, the cancer is further characterized by wild-type retinoblastoma protein and wild-type klotho beta.

In some embodiments, the cancer is characterized by wild-type retinoblastoma protein and wild-type klotho beta without statistically significant FGR19 overexpression or statistically significant FGR19 amplification.

In another aspect, the disclosure provides a method for treating a cancer (e.g., hepatocellular carcinoma) comprising administering to a subject a therapeutically effective amount of N-(2-((6-(2,6-dichloro-3,5-dimethoxyphenyl)quinazolin-2-yl)amino)-3-methylphenyl)acrylamide (Compound 2) or a pharmaceutically acceptable salt thereof in combination with 6-acetyl-8-cyclopentyl-5-methyl-2-{[5-(1-piperazinyl)-2-pyridinyl]amino}pyrido[2,3-d]pyrimidin-7(8H)-one (also referred to as palbociclib or PD0332991) or a pharmaceutically acceptable salt thereof.

In some embodiments, the cancer is hepatocellular carcinoma, breast cancer, ovarian cancer, lung cancer, liver cancer, a sarcoma, esophagus cancer, large intestine cancer, colon cancer, head and neck cancer, or hyperlipidemia. In some embodiments, the cancer is hepatocellular carcinoma. In some embodiments, the cancer is fibrolamellar hepatocellular carcinoma. In some embodiments, the cancer is fibrolamellar hepatocellular carcinoma. In some embodiments, the cancer is estrogen receptor (ER)-positive, human epidermal growth factor receptor 2 (HER2)-negative metastatic breast cancer.

In some embodiments, the cancer is pancreatic cancer (e.g., well or moderately differentiated metastatic pancreatic neuroendocrine tumors (pNET)), leukemia (e.g., acute myeloid leukemia or acute lymphoblastic leukemia), oligoastrocytoma, oligodendroglioma, liposarcoma, urothelial cancer, non-small cell lung cancer, squamous cell lung cancer, glioblastoma, thymic cancer, prostate cancer, esophagus cancer, large intestine cancer, colon cancer, head and neck cancer, or chordoma. In some embodiments, the cancer is advanced.

In some embodiments, the cancer is characterized by progressive brain metastases or recurrent, progressive, or refractory central nervous system tumors.

In some embodiments, 50 to 150 mg of palbociclib or an equivalent amount of a pharmaceutically acceptable salt of palbociclib is orally administered to the patient once daily. In some embodiments, 125 mg of palbociclib or an equivalent amount of a pharmaceutically acceptable salt of palbociclib is orally administered to the patient once daily. In some embodiments, less than 125 mg of palbociclib or an equivalent amount of a pharmaceutically acceptable salt of palbociclib is orally administered to the patient once daily.

In some embodiments, palbociclib is taken with food, optionally in combination with letrozole 2.5 mg once daily. In some embodiments, palbociclib is administered to the patient for twenty-one consecutive days, followed by seven days in which no palbociclib is administered to the patient. In some embodiments, the twenty-eight day administration schedule is repeated one or more times.

In some embodiments, the FGFR4 inhibitors of the disclosure inhibit FGFR4 activity more potently than they inhibit FGFR1 activity. For example, the FGFR4 inhibitors of the disclosure can inhibit FGFR4 activity at least 10 times, at least 50 times, at least 100 times, at least 200 times, or at least 500 times more potently than they inhibit FGFR1 activity.

In some embodiments, selectivity is measured by comparing the inhibition of FGFR1 and FGFR4 caused by the compound of this disclosure in the same type of assay. In some embodiments, the assays used to measure inhibition of FGFR1 and FGFR4 are any of the assays described herein. Typically, inhibition is expressed as IC₅₀ (the concentration of inhibitor at which 50% of the activity of the enzyme is inhibited) and thus fold-selectivity is measured by the equation:

$$\frac{{IC}_{50} FGFR 1}{{IC}_{50} FGFR 4}$$

Sensitivity to an inhibitor can also be expressed as EC₅₀ (the half maximal inhibitory concentration, GI₅₀ (the concentration of drug required to inhibit 50% of cell viability), or AUC (area under the curve, which provides a cumulative response metric). The same measurements and calculations can be used to measure selectivity over FGFR2 and FGFR3 as well.

Any other assays of FGFR activity may be utilized to determine the relative inhibition of FGFR1 and FGFR4 by the compounds of this disclosure as long as such assays utilize what one of skill in the art would deem to be the same parameters in measuring FGFR activity.

In another aspect, the disclosure provides a combination therapy comprising at least one selective fibroblast growth factor receptor 4 (FGFR4) inhibitor and at least one cyclin-dependent kinase 4/6 (CDK4/6) inhibitor.

In some embodiments, the at least one selective FGFR4 inhibitor is chosen from selective covalent FGFR4 inhibitors that covalently bind to Cys552 of FGFR4.

In some embodiments, the at least one selective FGFR4 inhibitor is chosen from compounds and pharmaceutically acceptable salts thereof as disclosed in U.S. Pat. Nos. 8,802,697, 9,266,883, 9,321,786, and 9,533,988, each of which is incorporated herein by reference in its entirety.

In some embodiments, the at least one selective FGFR4 inhibitor is chosen from N-((3S,4S)-3-((6-(2,6-dichloro-3,5-dimethoxyphenyl)quinazolin-2-yl)amino)tetrahydro-2H-pyran-4-yl)acrylamide (Compound 1), N-(2-((6-(2,6-dichloro-3,5-dimethoxyphenyl)quinazolin-2-yl)amino)-3-methylphenyl)acrylamide (Compound 2), and pharmaceutically acceptable salts thereof.

In some embodiments, the at least one selective FGFR4 inhibitor is chosen from N-[2-[[6-[(2,6-dichloro-3,5-dimethoxyphenyl)carbamoyl-methylamino]pyrimidin-4-yl]amino]-5-(4-ethylpiperazin-1-yl)phenyl]prop-2-enamide (also referred to as H3B-6527), N-[5-cyano-4-(2-methoxyethylamino)pyridin-2-yl]-7-formyl-6-[(4-methyl-2-oxopiperazin-1-yl)methyl]-3,4-dihydro-2H-1,8-naphthyridine-1-carboxamide (also referred to as FGF401), and pharmaceutically acceptable salts thereof. In some embodiments, the at least one selective FGFR4 inhibitor is an FGFR4 monoclonal antibody (e.g., U3-1784).

In some embodiments, the at least one CDK4/6 inhibitor is chosen from compounds and pharmaceutically acceptable salts thereof as disclosed in U.S. Pat. No. 6,936,612, U.S. Patent Application Publication No. 2013/0035336, U.S. Patent Application Publication No. 2013/0150342, U.S. Patent Application Publication No. 2016/0002223, WO 2011/101409, and WO 2014/128588, each of which is incorporated herein by reference in its entirety.

In some embodiments, the at least one CDK4/6 inhibitor is chosen from 6-acetyl-8-cyclopentyl-5-methyl-2-{[5-(1-piperazinyl)-2-pyridinyl]amino}pyrido[2,3-d]pyrimidin-7(8H)-one (also referred to as palbociclib or PD0332991) and pharmaceutically acceptable salts thereof.

In another aspect, the disclosure provides a method of treating a cancer in a patient in need thereof comprising:

determining if, having determined if, or receiving information that the patient has a cancer characterized by at least one biomarker chosen from fibroblast growth factor 19 (FGF19) overexpression, amplified FGF19, and fibroblast growth factor receptor 4 (FGFR4) overexpression;

identifying the patient as responsive to a combination therapy described herein; and

administering a therapeutically effective amount of the combination therapy to the patient.

In another aspect, the disclosure provides a method of treating a cancer in a patient in need thereof comprising administering a therapeutically effective amount of a combination therapy described herein to a patient having a cancer characterized by at least one biomarker chosen from fibroblast growth factor 19 (FGF19) overexpression, amplified FGF19, and fibroblast growth factor receptor 4 (FGFR4) overexpression, wherein the cancer is responsive to the combination therapy.

### DETAILED DESCRIPTION

Some FGFR4 inhibitors disclosed herein can form a covalent bond with FGFR4. For example, some FGFR4 inhibitors disclosed herein can form a covalent bond with a cysteine residue of FGFR4 (e.g., the cysteine at residue 552 (Cys552)). FGFRs 1-3 do not contain this cysteine. The ability to form a covalent bond between the inhibitor and FGFR4 is an important factor in FGFR4 selectivity.

The details of construction and the arrangement of components set forth in the following description or illustrated in the drawings are not meant to be limiting. Other embodiments and different ways to practice the disclosure are expressly included. Also, the phraseology and terminology used herein are for the purpose of description and should not be regarded as limiting. The use of “including,” “includes,” “include,” “comprising,” “having,” “containing,” “involving,” and variations thereof is meant to encompass the items listed thereafter and equivalents thereof as well as additional items.

### Definitions

“Aliphatic group,” as used herein, refers to a straight-chain, branched-chain, or cyclic hydrocarbon group and includes saturated and unsaturated groups, such as an alkyl group, an alkenyl group, or an alkynyl group.

“Alkenyl,” as used herein, refers to an aliphatic group containing at least one double bond.

“Alkoxyl” or “alkoxy,” as used herein, refers to an alkyl group having an oxygen radical attached thereto. Representative alkoxyl groups include methoxy, ethoxy, propyloxy, tert-butoxy, and the like.

“Alkyl,” as used herein, refers to a monovalent radical of a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Representative alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3 -dimethyl-l-butyl, 2-ethyl-1-butyl, butyl, isobutyl, t-butyl, pentyl, isopentyl, neopentyl, hexyl, heptyl, octyl, etc.

“Alkylene,” as used herein, refers to a divalent radical of an alkyl group, e.g., —CH₂—, —CH₂CH₂—, and —CH₂CH₂CH₂—.

“Alkynyl,” as used herein, refers to a straight or branched hydrocarbon chain containing 2-12 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

“Alkynylene,” as used herein, refers to an alkynyl having two connecting points. For example, “ethynylene” represents the group Alkynylene groups can also be in an unsubstituted form or substituted form with one or more substituents.

“Alkylthio,” as used herein, refers to a hydrocarbyl group having a sulfur radical attached thereto. In some embodiments, the “alkylthio” moiety is represented by one of —S-alkyl, —S-alkenyl, or —S-alkynyl. Representative alkylthio groups include methylthio, ethylthio, and the like.

“Amido,” as used herein, refers to —C(═O)—N(R¹)(R²) or —N(R¹)—C(═O)—R², where each of R¹ and R² is H, alkyl, cycloalkyl, alkoxy, or hydroxy.

“Amino,” as used herein, refers to —NH₂, —NH(alkyl), or —N(alkyl)(alkyl).

“Amplified,” as used herein, means additional copies of a gene or chromosome segment are produced in cancer cells that may confer a growth or survival advantage. One skilled in the art could measure the number of copies of a gene or chromosome segment using techniques routine in the art, such as, for example, fluorescent in situ hybridization (FISH) comparative genomic hybridization and with high-resolution array-based tests based on array comparative genomic hybridization (or aCGH), SNP array technologies and high resolution microarrays that include copy number probes as well an SNPs as well as whole genome (WGS) or whole exome DNA sequencing (WES) using next generation sequencing (NGS) technologies.

“Arylalkyl” or “aralkyl,” as used herein, refers to an alkyl group substituted with an aryl group (e.g., an aromatic or heteroaromatic group). Aralkyl includes groups in which more than one hydrogen atom has been replaced by an aryl group. Non-limiting examples of “arylalkyl” or “aralkyl” include benzyl, 2-phenylethyl, 3-phenylpropyl, 9-fluorenyl, benzhydryl, and trityl groups.

“Aryl,” as used herein, refers to 5-, 6-, and 7-membered single-ring aromatic groups that may include from zero to four heteroatoms, for example, phenyl, pyrrolyl, furanyl, thiophenyl, imidazolyl, oxazolyl, thiazolyl, triazolyl, pyrazolyl, pyridinyl, pyrazinyl, pyridazinyl, pyrimidinyl, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, polycyclyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls, and/or heterocyclyls. Each ring can contain, e.g., five to seven members.

“Carbocyclic ring system,” as used herein, refers to a monocyclic, bicyclic, or polycyclic hydrocarbon ring system, wherein each ring is either completely saturated or contains one or more units of unsaturation, but where no ring is aromatic.

“Carbocyclyl,” as used herein, refers to a monovalent radical of a carbocyclic ring system. Representative carbocyclyl groups include cycloalkyl groups (e.g., cyclopentyl, cyclobutyl, cyclopentyl, cyclohexyl, and the like) and cycloalkenyl groups (e.g., cyclopentenyl, cyclohexenyl, cyclopentadienyl, and the like).

“Cycloalkyl,” as used herein, refers to a cyclic, bicyclic, tricyclic, or polycyclic non-aromatic hydrocarbon groups having three to twelve carbons. Any substitutable ring atom can be substituted (e.g., by one or more substituents). The cycloalkyl groups can contain fused or spiro rings. Fused rings are rings that share a common carbon atom. Examples of cycloalkyl moieties include, but are not limited to, cyclopropyl, cyclohexyl, methylcyclohexyl, adamantyl, and norbornyl.

“Cycloalkylalkyl,” as used herein, refers to a -(cycloalkyl)-alkyl radical where cycloalkyl and alkyl are as disclosed herein. The “cycloalkylalkyl” is bonded to the parent molecular structure through the cycloalkyl group.

“Cyano,” as used, herein refers to —CN.

“Covalent inhibitor,” as used herein, means an inhibitor that can form a covalent bond with a protein.

“Ester” as used herein refers to —C(═O)—O(R¹) or —O—C(═O)—R¹, wherein R¹ is H or alkyl.

“FGFR4” or “FGFR4 protein,” as used herein, refers to any form of the FGFR4 protein, including wild-type and all variant forms (including, without limitation, mutant forms and splice variants). The FGFR4 protein is a product of the FGFR4 gene, and the FGFR4 protein therefore includes any protein encoded by any form of the FGFR4 gene, including any aberrations, e.g., point mutations, indels, translocation fusions, and focal amplifications.

“Heteroaromatic ring system” is art-recognized and refers to a monocyclic, bicyclic, or polycyclic ring system wherein at least one ring is both aromatic and comprises at least one heteroatom (e.g., N, O, or S); and wherein no other rings are heterocyclyl (as defined below). In certain instances, a ring which is aromatic and comprises a heteroatom contains one, two, three, or four ring heteroatoms in such ring.

“Heteroaryl,” as used herein, refers to a monovalent radical of a heteroaromatic ring system. Representative heteroaryl groups include ring systems where (i) each ring comprises a heteroatom and is aromatic, e.g., imidazolyl, oxazolyl, thiazolyl, triazolyl, pyrrolyl, furanyl, thiophenyl pyrazolyl, pyridinyl, pyrazinyl, pyridazinyl, pyrimidinyl, indolizinyl, purinyl, naphthyridinyl, pyrido[2,3-d]pyrimidine, and pteridinyl; (ii) each ring is aromatic or carbocyclyl, at least one aromatic ring comprises a heteroatom and at least one other ring is a hydrocarbon ring or e.g., indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, pyrido[2,3-b]-1,4-oxazin-3-(4H)-one, 5,6,7,8-tetrahydroquinolinyl, and 5,6,7,8-tetrahydroisoquinolinyl; and (iii) each ring is aromatic or carbocyclyl, and at least one aromatic ring shares a bridgehead heteroatom with another aromatic ring, e.g., 4H-quinolizinyl.

“Heterocyclic ring system,” as used herein, refers to monocyclic, bicyclic, and polycyclic ring systems where at least one ring is saturated or partially unsaturated (but not aromatic) and comprises at least one heteroatom. A heterocyclic ring system can be attached to its pendant group at any heteroatom or carbon atom that results in a stable structure and any of the ring atoms can be optionally substituted.

“Heterocyclyl,” as used herein, refers to a monovalent radical of a heterocyclic ring system. Representative heterocyclyls include ring systems in which (i) every ring is non-aromatic and at least one ring comprises a heteroatom, e.g., tetrahydrofuranyl, tetrahydropyranyl, tetrahydrothienyl, pyrrolidinyl, pyranyl, thianyl, pyrrolidonyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, and quinuclidinyl; (ii) at least one ring is non-aromatic and comprises a heteroatom and at least one other ring is an aromatic carbon ring, e.g., 1,2,3,4-tetrahydroquinolinyl or 1,2,3,4-tetrahydroisoquinolinyl; and (iii) at least one ring is non-aromatic and comprises a heteroatom and at least one other ring is aromatic and comprises a heteroatom, e.g., 3,4-dihydro-1H-pyrano[4,3-c]pyridine or 1,2,3,4-tetrahydro-2,6-naphthyridine.

In some embodiments, a heterocyclyl is chosen from:

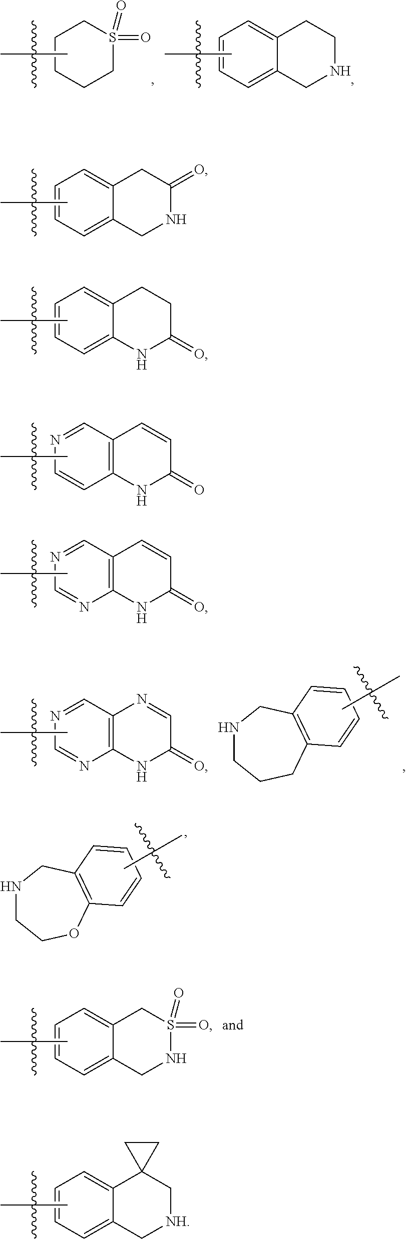

“Heterocyclylalkyl,” as used herein, refers to an alkyl group substituted with a heterocyclyl group.

“Heteroarylalkyl,” as used herein, refers to an alkyl group substituted with a heteroaryl group.

“Hydroxy” or “hydroxyl,” as used herein, refers to —OH.

“Inhibitor,” as used herein, refers to a compound or antibody that inhibits an enzyme such that a reduction in activity of the enzyme can be observed, e.g., in a biochemical assay. In certain embodiments, an inhibitor has an IC₅₀ of less than 1 μM, less than 500 nM, less than 250 nM, less than 100 nM, less than 50 nM, or less than 10 nM. An FGFR4 inhibitor refers to a compound that inhibits FGFR4; a CDK inhibitor refers to a compound or antibody that inhibits a CDK.

“Nitro,” as used herein, refers to —NO₂.

“Nucleophile,” as used herein, refers to a species that donates an electron pair to an electrophile to form a chemical bond in a reaction. In some embodiments, a nucleophile can be: an oxygen nucleophile, e.g., water or hydroxyl; a nitrogen nucleophile, e.g., amine; or a sulfur nucleophile, e.g., thiol, such as, for example, the thiol in the side chain of a cysteine residue.

“Overexpressed,” as used herein, means there is production of a gene product in a sample that is higher than that observed in a population of control samples (e.g., normal tissue). Overexpression encompasses expression if the gene product ordinarily is not produced in control samples. Production of a gene product may be measured using routine techniques in the art, such as, for example, immunohistochemistry. In one aspect, overexpression of FGF19 gene product is ≥1% expression of FGF19 protein.

“Selective” refers to a compound that inhibits the activity of a target protein, e.g., FGFR4, more potently than it inhibits the activity of other proteins. In this instance, the isoforms FGFR1, FGFR2, FGFR3, and FGFR4 are all considered distinct proteins. In some embodiments, a compound can inhibit the activity of the target protein, e.g., FGFR4, at least 1.5, at least 2, at least 5, at least 10, at least 20, at least 30, at least 40, at least 50, at least 60, at least 70, at least 80, at least 90, at least 100, at least 200, at least 500, or at least 1000 or more times potently than it inhibits the activity of a non-target protein.

“Substituted,” whether preceded by the term “optionally” or not, refers herein to moieties having substituents replacing a hydrogen on one or more carbons of the backbone. One skilled in the art will be understand that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, and aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. Those skilled in the art will understand that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl, and sulfonate), and silyl groups, as well as ethers, alkylthios, carbonyls (including ketones, aldehydes, carboxylates, and esters), —CF₃, —CN, and the like. Example substituted alkyls are described below. Cycloalkyls can be further substituted with alkyls, alkenyls, alkoxys, alkylthios, aminoalkyls, carbonyl-substituted alkyls, —CF₃, —CN, and the like. Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls, or alkynyls.

As used herein, the definition of each expression, e.g., alkyl, m, n, etc., when it occurs more than once in any structure, is intended to be independent of its definition elsewhere in the same structure.

“Sulfonyl” as used herein refers to —SO₂—.

“Sulfonamido” as used herein refers to —S(═O)—N(R¹)(R²) or —N(R¹)—S(═O)—R², wherein each of R¹ and R² is independently H or alkyl.

“Warhead moiety” or “warhead” refers to a moiety of an inhibitor which participates, either reversibly or irreversibly, with the reaction of a donor, e.g., a protein, with a substrate. Warheads may, for example, form covalent bonds with the protein, or may create stable transition states, or be a reversible or an irreversible alkylating agent. For example, the warhead moiety can be a functional group on an inhibitor that can participate in a bond-forming reaction, wherein a new covalent bond is formed between a portion of the warhead and a donor, for example an amino acid residue of a protein. The warhead is an electrophile and the “donor” is a nucleophile, such as the sulfur atom of a cysteine residue. Examples of suitable warheads include, without limitation, the following groups:

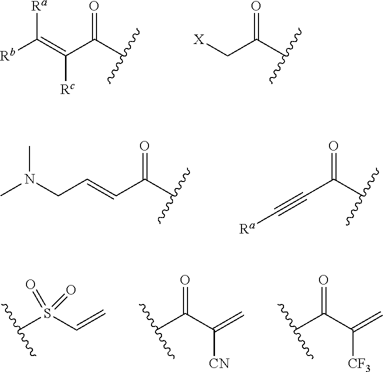

wherein X is a leaving group, such as halo, or an activated hydroxyl moiety (e.g., triflate); and

each of R^{a}, R^{b}, and R^{c} is, independently, H, substituted or unsubstituted C₁₋₄ alkyl, substituted or unsubstituted C₃₋₄ cycloalkyl, or cyano.

As used herein, the terms “patient,” “subject,” “individual,” and “host” refer to either a human or a non-human animal suffering from or suspected of suffering from a disease or disorder, e.g., a cancer mediated by FGFR4 or CDK4/6.

“Treat” and “treating” such a disease or disorder refers to ameliorating at least one symptom of the disease or disorder. These terms, when used in connection with a disease such as a cancer, refer to one or more of: impeding growth of the cancer; causing the cancer to shrink by weight or volume; extending the expected survival time of the patient; inhibiting tumor growth; reducing tumor mass; reducing size or number of metastatic lesions; inhibiting the development of new metastatic lesions; prolonging survival; prolonging progression-free survival; prolonging time to progression; and/or enhancing quality of life.

The term “therapeutic effect” refers to a beneficial local or systemic effect in animals, for example mammals, such as, for example, humans, caused by administration of a compound or combination of the disclosure. The phrase “therapeutically effective amount” means that amount of a compound or combination of the disclosure that is effective to treat a disease or disorder at a reasonable benefit/risk ratio. The therapeutically effective amount of the compound or combination will vary depending upon the subject and disease or disorder being treated, the weight and age of the subject, the severity of the disease or disorder, the manner of administration, and the like, which can readily be determined by one of skill in the art.

The phrase “combination therapy” as used herein refers to a dosing regimen that requires administration of at least two different compounds (e.g., at least one FGFR4 inhibitor and at least one CDK4/6 inhibitor) to a patient. The compounds may be administered simultaneously or at different times in a single day. The dosing regimens for the at least two compounds may, but is not required, to overlap.

The phrase “total daily dose” as used herein refers to the amount of a compound administered to a subject in a twenty-four hour time window.

The term “co-administering” as used herein means exposing a subject to two or more therapeutic regimens (e.g., two or more compounds) simultaneously. In some embodiments, two or more compounds may be administered simultaneously; in some embodiments, such compounds may be administered sequentially; in some embodiments, such compounds are administered in overlapping dosing regimens. In some embodiments, “administration” of combination therapy may involve administration of one or more compounds to a subject already receiving the other compound(s). For clarity, a combination therapy does not require that individual compounds be administered together in a single composition (or even necessarily at the same time), although in some embodiments, two or more compounds may be administered together in a single combination. In some embodiments, the compounds to be co-administered are in separate dosage forms, but packaged together (e.g., in a blister pack or other pharmaceutical kit) so as to facilitate their co-administration.

The compounds described herein may contain unnatural proportions of atomic isotopes at one or more of the atoms that constitute such compounds. For example, the compounds may be radiolabeled with radioactive isotopes, such as, for example, tritium (³H) or carbon-14 (¹⁴C). All isotopic variations of the compounds disclosed herein, whether radioactive or not, are intended to be encompassed within the scope of the present disclosure. For example, deuterated compounds or compounds containing ¹³C are intended to be encompassed within the scope of the disclosure.

Certain compounds can exist in different tautomeric forms, and all possible tautomeric forms of all of the compounds described herein are intended to be encompassed within the scope of the disclosure.

The “enantiomeric excess” or “% enantiomeric excess” of a composition can be calculated using the equation shown below. In the example shown below, a composition contains 90% of one enantiomer, e.g., the S-enantiomer, and 10% of the other enantiomer, e.g., the R-enantiomer.

$$ee = \frac{90 - 10}{100} = 80 \%$$

Thus, a composition containing 90% of one enantiomer and 10% of the other enantiomer is said to have an enantiomeric excess of 80%. Some of the compositions described herein contain an enantiomeric excess of at least 50%, at least 75%, at least 80%, at least 85%, at least 90%, at least 95%, or at least 99% of Compound 1 (the S-enantiomer). In other words, the compositions contain an enantiomeric excess of the S-enantiomer over the R-enantiomer.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure.

The compounds described herein can be useful as the free base or as a salt. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts. See, e.g., Berge et al. (1977) “Pharmaceutical Salts”, J. Pharm. Sci. 66:1-19.

Certain compounds disclosed herein can exist in unsolvated forms as well as solvated forms, including hydrated forms. In general, the solvated forms are equivalent to unsolvated forms and are encompassed within the scope of the present disclosure. Certain compounds disclosed herein may exist in multiple crystalline or amorphous forms. In general, all physical forms are equivalent for the uses contemplated by the present disclosure and are intended to be within the scope of the present disclosure.

### Combination Therapy

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two (or more) treatments can be partially additive, wholly additive, or greater than additive. The delivery can be such that an effect of the first treatment delivered is still detectable when the second is delivered.

The at least one FGFR4 inhibitor described herein and the at least one CDK inhibitor (e.g., the at least one CDK4/6 inhibitor) can be administered simultaneously, in the same or in separate compositions, or sequentially. For sequential administration, the at least one FGFR4 inhibitor described herein can be administered first, and the at least one CDK inhibitor (e.g., the at least one CDK4/6 inhibitor) can be administered second, or the order of administration can be reversed.

In some embodiments, the combination therapy provides increased progression-free survival (PFS) in comparison to monotherapy by about 2 months, about 4 months, about 6 months, about 8 months, about 10 months, about 1 year, about 1.5 years, about 2 years, or about more than 2 years. In some embodiments, the combination therapy delays the emergence of resistance by about 2 months, about 4 months, about 6 months, about 8 months, about 10 months, about 1 year, about 1.5 years, about 2 years, or about more than 2 years.

### Pharmaceutical Compositions

While it is possible for a compound disclosed herein to be administered alone, it is preferable to administer the compound as a pharmaceutical formulation, where the compound is combined with one or more pharmaceutically acceptable excipients or carriers. The compounds disclosed herein may be formulated for administration in any convenient way for use in human or veterinary medicine. In some embodiments, the compound included in the pharmaceutical preparation may be active itself, or may be a prodrug, e.g., capable of being converted to an active compound in a physiological setting. In some embodiments, the compounds provided herein include their hydrates.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Examples of pharmaceutically acceptable salts of a compound described herein include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, and undecanoate. Salts derived from appropriate bases include alkali metal (e.g., sodium), alkaline earth metal (e.g., magnesium), ammonium, and N-(alkyl)₄⁺ salts. This disclosure also envisions the quaternization of any basic nitrogen-containing groups of the compounds described herein. Water or oil soluble or dispersible products may be obtained by such quaternization.

Examples of pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) cyclodextrins, such as Captisol®, targeting ligands attached to nanoparticles, such as Accurins™; and (22) other non-toxic compatible substances, such as polymer-based compositions, employed in pharmaceutical formulations.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Solid dosage forms (e.g., capsules, tablets, pills, dragees, powders, granules, and the like) can include one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents.

Liquid dosage forms can include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, fatty acid esters of sorbitan, and mixtures thereof.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

Ointments, pastes, creams, and gels may contain, in addition to an active compound, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to an active compound, excipients such as lactose, talc, silicic acid, aluminum hydroxide, calcium silicates, and polyamide powder, or mixtures thereof. Sprays can additionally contain customary propellants, such as chlorofluorohydrocarbons and volatile unsubstituted hydrocarbons, such as butane and propane.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect.

Dosage forms for the topical or transdermal administration of a compound described herein include powders, sprays, ointments, pastes, creams, lotions, gels, solutions, patches, and inhalants. The active compound may be mixed under sterile conditions with a pharmaceutically acceptable carrier, and with any preservatives, buffers, or propellants that may be required.

When the compounds disclosed herein are administered as pharmaceuticals to humans or animals, they can be given per se or as a pharmaceutical composition containing, for example, 0.1% to 99.5% (for example, 0.5% to 90%) of active ingredient in combination with a pharmaceutically acceptable carrier.

The formulations can be administered topically, orally, transdermally, rectally, vaginally, parentally, intranasally, intrapulmonary, intraocularly, intravenously, intramuscularly, intraarterially, intrathecally, intracapsularly, intradermally, intraperitoneally, subcutaneously, subcuticularly, or by inhalation.

### Indications

FGFR4 regulates proliferation, survival, and alpha-fetoprotein secretion during hepatocellular carcinoma (HCC) progression. Inhibitors of FGFR4 are therefore promising potential therapeutic agents for this unmet medical need (Ho et al., Journal of Hepatology, 2009, 50:118-27). HCC afflicts more than 700,000 people worldwide every year and has one of the worst one-year survival rates of any cancer type (Torre et al—2015—CA A Cancer Journal for Clinicians; Bruix et al (2016) ESMO World GI Abstracts; Llovet J et al NEJM 2008 359:378-390; Cheng Ann-li et al Lancet 2009 10:25-33. Further evidence of the link between FGFR4 and HCC is shown through the involvement of FGF19, a member of the fibroblast growth factor (FGF) family, which consists of hormones that regulate glucose, lipid, and energy homeostasis. Increased hepatocyte proliferation and liver tumor formation have been observed in FGF19 transgenic mice. FGF19 activates FGFR4, its predominant receptor in the liver, and it is believed that activation of FGFR4 is the mechanism whereby FGF19 can increase hepatocyte proliferation and induce hepatocellular carcinoma formation (Wu et al., J Biol Chem (2010) 285(8):5165-5170). FGF19 has also been identified as a driver gene in HCC by other groups (Sawey et al., Cancer Cell (2011) 19: 347-358).

Compound 2, a selective FGFR4 inhibitor, is known to inhibit proliferation in HCC cell lines with an intact FGFR4 signaling pathway (FGFR4, FGF19, and KLB) (Hagel et al., Cancer Discovery (2015) 425-37). In addition, CCND1, which co-activates CDK4/6, and FGF19 are co-amplified in HCC patients and may contribute to liver tumorigenesis. It is therefore believed that the combination therapies disclosed herein can be used to treat HCC and other liver cancers.

Oncogenome screening has identified an activating fibroblast growth factor receptor 4 (FGFR4) Y367C mutation in the human breast cancer cell line MDA-MB-453. This mutation was shown to elicit constitutive phosphorylation, leading to an activation of the mitogen-activated protein kinase cascade. Accordingly, it has been suggested that FGFR4 may be a driver of tumor growth in breast cancer (Roidl et al., Oncogene (2010) 29(10):1543-1552). It is therefore believed that the combination therapies disclosed herein can be used to treat FGFR4 modulated breast cancer. Molecular changes (e.g., translocations) in genes upstream of FGFR4 can lead to activation or overexpression of FGFR4. For example, a PAX3-FKHR translocation/gene fusion can lead to FGFR4 overexpression.

Overexpression of FGFR4 due to this mechanism has been associated with rhabdomyosarcoma (RMS) (Cao et al., Cancer Res (2010) 70(16): 6497-6508). Mutations in FGFR4 itself (e.g., kinase domain mutations) can lead to over-activation of the protein; this mechanism has been associated with a subpopulation of RMS (Taylor et al., J Clin Invest (2009) 119: 3395-3407). It is therefore believed that the combination therapies disclosed herein can be used to treat FGFR4 modulated RMS and other sarcomas.

Other diseases have been associated with changes in genes upstream of FGFR4 or with mutations in FGFR4 itself. For example, mutations in the kinase domain of FGFR4 lead to over-activation, which has been associated with lung adenocarcinoma (Ding et al., Nature (2008) 455(7216): 1069-1075). Amplification of FGFR4 has been associated with conditions such as renal cell carcinoma. In addition, silencing FGFR4 and inhibiting ligand-receptor binding significantly decrease ovarian tumor growth, suggesting that the combination therapies disclosed herein could be useful in treating ovarian cancer (Zaid et al., Clin. Cancer Res. (2013) 809).

Pathogenic elevations of bile acid levels have been linked to variations in FGF19 levels (Vergnes et al., Cell Metabolism (2013) 17, 916-28). Reduction in the level of FGF19 may therefore be beneficial in promoting bile acid synthesis and thus in treating hyperlipidemia. It is therefore believed that the combination therapies disclosed herein can be used to treat hyperlipidemia.

Cyclin dependent kinases, such as CDK4/6, are critical for cell division and proliferation regulation. Increased activity or temporally abnormal activation of CDKs can lead to tumor formation in humans. For example, alterations in CDKs or their regulators are commonly associated with tumor development. CDK inhibitors such as p16 and p27 can inhibit in vitro lung cancer cell growth (Kamb, A., Curr. Top. Microbiol. Immunol. (1998) 227, 139-148).

CDK inhibitors, such as cyclin dependent kinase 4/6 (CDK4/6) inhibitors, may be useful for reducing cancer cell proliferation mediated at least in part by an activated CDK pathway (e.g., an activated CDK4/6 pathway). Illustratively, CDK inhibitors may be useful in the treatment of tumors with amplifications of CDK genes (e.g., CDK4 and CDK6 genes), as well as tumors overexpressing cyclin partners of the CDKs. CDK inhibitors may also be useful for treating cancers associated with D-cyclin translocations (e.g., mantle cell lymphoma or multiple myeloma), D-cyclin amplifications (e.g., breast cancer or squamous cell esophageal cancer), CDK4 amplifications (e.g., liposarcoma), CDK6 amplifications or overexpressions (e.g., T-cell lymphoma), or p16 inactivation (e.g., melanoma, non-small cell lung cancer, or pancreatic cancer). In addition, CDK inhibitors may be useful for treating other diseases in which the underlying pathology is mediated, at least in part, by a CDK (e.g., CDK4/6), including diseases characterized by cell proliferation, apoptosis, or differentiation.

### Dose Levels

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound disclosed herein employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health, and prior medical history of the patient being treated, and like factors well-known in the medical arts.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In general, a suitable daily dose of a compound of the disclosure will be that amount of the compound that is the lowest dose effective to produce a therapeutic effect. Such an effective dose will generally depend upon the factors described above. Generally, doses of the compounds of this disclosure for a patient will range from 0.0001 mg to 100 mg per kilogram of body weight per day. For example, the dose could be between 10 mg and 2000 mg per day. Alternatively, the dose can be between 100 and 1000 mg per day, or between 200 and 600 mg per day. If desired, the effective daily dose of the active compound may be administered as one, two, three, four, or more sub-doses administered separately at appropriate intervals throughout the day, optionally, in unit dosage forms. In certain embodiments, the dosage of each of at least one FGFR4 inhibitor and the at least one CDK inhibitor are equal to the dose of each inhibitor when used in a monotherapy.

As demonstrated herein, the combination of at least one FGFR4 inhibitor and at least one CDK inhibitor (e.g., at least CDK4/6 inhibitor) in the treatment of cancer show unexpected synergy. Because of that synergy, it may be possible to use dosages of FGFR4 inhibitor and/or CDK inhibitor that are less than those used in a monotherapy. Accordingly, in some embodiments, the dosage of the FGFR4 inhibitor used in the methods of this disclosure is less than 95%, less than 90%, less than 85%, less than 80%, less than 75%, or less than 70% of the dose used when the FGFR4 inhibitor is used as a monotherapy. In other embodiments, the dosage of the CDK inhibitor (e.g., CDK4/6 inhibitor) used in the methods of this disclosure is less than 95%, less than 90%, less than 85%, less than 80%, less than 75%, or less than 70% of the dose used when the CDK inhibitor is used as a monotherapy. In still other embodiments, both the dosage of the FGFR4 inhibitor and the CDK inhibitor used in the methods of this disclosure is less than 95%, less than 90%, less than 85%, less than 80%, less than 75%, or less than 70% of the dose used when each of such inhibitors is used as a monotherapy.

As a non-limiting example, the dose commonly used for palbociclib as a monotherapy is 125 mg once daily. In some embodiments, the total daily dose of palbociclib or an equivalent amount of a pharmaceutically acceptable salt of palbociclib is less than 125 mg once daily, such as, for example, 25 mg, 30 mg, 35 mg, 40 mg, 45 mg, 50 mg, 55 mg, 60 mg, 70 mg, 75 mg, 80 mg, 85 mg, 90 mg, 95 mg, 100 mg, 105 mg, 110 mg, 115 mg, or 120 mg once daily.

In some embodiments, palbociclib or a pharmaceutically acceptable salt thereof is administered with food.

In some embodiments, palbociclib is administered to the patient for twenty-one consecutive days, followed by seven days in which no palbociclib is administered to the patient. In some embodiments, the twenty-eight day administration schedule is repeated one or more times.

In some embodiments, Compound 1 or a pharmaceutically acceptable salt thereof is orally administered to a patient once day (qd schedule) or twice daily (bid schedule).

In some embodiments, Compound 1 or a pharmaceutically acceptable salt thereof is orally administered to the patient once daily (qd schedule). In some embodiments, up to 600 mg of Compound 1 or a pharmaceutically acceptable salt thereof of Compound 1 is administered once daily.

In some embodiments, 100 mg, 140 mg, 280 mg, 420 mg, or 600 mg of Compound 1 or an equivalent amount of a pharmaceutically acceptable salt of Compound 1 is administered once daily. In some embodiments, Compound 1 is administered in the form of a tablet.

In some embodiments, Compound 1 or a pharmaceutically acceptable salt thereof is orally administered to the patient twice daily (bid schedule).

In some embodiments, 100 mg to 300 mg of Compound 1 or an equivalent amount of a pharmaceutically acceptable salt of Compound 1 is administered twice daily.

In some embodiments, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, or 500 mg, of Compound 1 or an equivalent amount of a pharmaceutically acceptable salt of Compound 1 is administered twice daily. In some embodiments, 100 mg, 150 mg, 200 mg, or 300 mg of Compound 1 or an equivalent amount of a pharmaceutically acceptable salt of Compound 1 is administered twice daily. In some embodiments, Compound 1 is administered in the form of a tablet.

In some embodiments, 100 mg of Compound 1 or an equivalent amount of a pharmaceutically acceptable salt thereof is administered twice daily.

In some embodiments, the total daily dose of Compound 1 or an equivalent amount of a pharmaceutically acceptable salt thereof is less than 600 mg, less than 400 mg, less than 300 mg, or less than 200 mg.

In some embodiments, Compound 1 is administered once in the morning and once in the evening. In some embodiments, the time between administering the doses is approximately ten to fourteen hours. In some embodiments, the time between administering the doses is at least eight hours.

In some embodiments, the patient administered Compound 1 twice daily (bid schedule) has reduced side effects compared to a patient administered Compound 1 once daily (qd schedule), e.g., reduced diarrhea, reduced nausea, reduced vomiting, reduced ALT increase, reduced AST increase, and/or reduced abdominal pain.

In some embodiments, the patient administered Compound 1 twice day (bid schedule) has improved efficacy compared to a patient administered Compound 1 once day (qd schedule) e.g., improved median time to progression (TTP), improved three- and six-month progression-free survival (PFS), and/or improved overall survival (OS).

### Pharmaceutical Kits

In some embodiments, the disclosure provides a kit (e.g., a pharmaceutical pack) comprising at least one inhibitor FGFR4 inhibitor and at least one CDK inhibitor (e.g., at least one CDK4/6 inhibitor), wherein the at least one FGFR4 inhibitor and the at least one CDK inhibitor are each formulated into separate dosage forms. The kits are useful for treating a cancer described herein. The kits may comprise at least one FGFR4 inhibitor and at least one CDK inhibitor in a separate container (e.g., a vial, ampule, bottle, syringe, and/or dispenser package, or other suitable container). In some embodiments, the provided kits may optionally further include additional containers comprising a pharmaceutical excipient for dilution or suspension of one or more of the at least one FGFR4 inhibitor and the at least one CDK inhibitor. In some embodiments, the at least one FGFR4 inhibitor and at least one CDK inhibitor in separate containers are combined (optionally in a third container comprising a pharmaceutical excipient for dilution or suspension) to form one unit dosage form prior to administration.

The kit may further include written instructions for administration of the inhibitors (e.g., how to combine the inhibitors into a single dosage form, the types of cancer for which the kit is useful, the frequency of administration of each inhibitor as separate dosage forms, and other information relevant to the co-administration of the inhibitors).

### Certain Embodiments

1. A method for treating a cancer in a patient in need thereof comprising administering a therapeutically effective amount of at least one fibroblast growth factor receptor 4 (FGFR4) inhibitor in combination with at least one cyclin-dependent kinase (CDK) inhibitor to the patient.

2. The method of embodiment 1, wherein the at least one CDK inhibitor is chosen from CDK4/6 inhibitors.

3. The method of embodiment 1 or 2, wherein the at least one CDK inhibitor is chosen from palbociclib, 7-cyclopentyl-N,N-dimethyl-2-((5-(piperazin-1-yl)pyridin-2-yl)amino)-7H-pyrrolo[2,3-d]pyrimidine-6-carboxamide, 2-(2-chlorophenyl)-5,7-dihydroxy-8-[(3S,4R)-3-hydroxy-1-methyl-4-piperidinyl]-4-chromenone, N-(5-((4-ethylpiperazin-1-yl)methyl)pyridin-2-yl)-5-fluoro-4-(4-fluoro-1-isopropyl-2-methyl-1H-benzo[d]imidazol-6-yl)pyrimidin-2-amine, GZ38-1, and pharmaceutically acceptable salts thereof.

4. The method of any one of embodiments 1 to 3, wherein the at least one CDK inhibitor is chosen from palbociclib and pharmaceutically acceptable salts thereof.

5. The method of any one of embodiments 1 to 4, wherein the at least one FGFR4 inhibitor is chosen from selective FGFR4 inhibitors.

6. The method of any one of embodiments 1 to 5, wherein the at least one FGFR4 inhibitor is chosen from selective covalent FGFR4 inhibitors that covalently bind to Cys552 of FGFR4.

7. The method of any one of embodiments 1 to 6, wherein the at least one FGFR4 inhibitor is chosen from N-((3S,4S)-3-((6-(2,6-dichloro-3,5-dimethoxyphenyl)quinazolin-2-yl)amino)tetrahydro-2H-pyran-4-yl)acrylamide (Compound 1), N-(2-((6-(2,6-dichloro-3,5-dimethoxyphenyl)quinazolin-2-yl)amino)-3-methylphenyl)acrylamide (Compound 2), and pharmaceutically acceptable salts thereof.

8. The method of any one of embodiments 1 to 7, wherein the cancer is characterized by fibroblast growth factor 19 (FGF19) overexpression.

9. The method of any one of embodiments 1 to 9, wherein the cancer is characterized by amplified FGF19.

10. The method of embodiment 9, wherein the cancer is further characterized by wild-type retinoblastoma protein and wild type klotho beta.

11. The method of any one of embodiments 1 to 10, wherein the cancer is characterized by amplified FGF19 and FGF19 overexpression.

12. The method of any one of embodiments 1 to 8, wherein the cancer is characterized by FGR19 overexpression without statistically significant FGR19 amplification.

13. The method of any one of embodiments 1 to 7, wherein the cancer is characterized by wild-type retinoblastoma protein and wild-type klotho beta without statistically significant FGR19 overexpression or statistically significant FGR19 amplification.

14. The method of any one of embodiments 1 to 13, wherein the cancer is characterized by FGFR4 overexpression.

15. The method of any one of embodiments 1 to 14, wherein the cancer is breast cancer, ovarian cancer, lung cancer, liver cancer, a sarcoma, esophagus cancer, large intestine cancer, colon cancer, head and neck cancer, or hyperlipidemia.

16. The method of embodiment 15, wherein the cancer is liver cancer.

17. The method of embodiment 16, wherein the cancer is hepatocellular carcinoma or hepatoblastoma.

18. The method of embodiment 17, wherein the cancer is fibrolamellar hepatocellular carcinoma.

19. The method of embodiment 17, wherein the cancer is unresectable hepatocellular carcinoma.

20. The method of embodiment 15, wherein the cancer is breast cancer.

21. The method of embodiment 20, wherein the cancer is metastatic breast cancer.

22. The method of embodiment 21, wherein the cancer is receptor (ER)-positive, human epidermal growth factor receptor 2 (HER2)-negative metastatic breast cancer.

23. The method of any one of embodiments 1 to 22, wherein the patient is a human.

24. The method of any one of embodiments 1 to 23, wherein the patient has been previously treated with a tyrosine kinase inhibitor e.g., sorafenib.

25. The method of any one of embodiments 1 to 23, wherein the patient has not been previously treated with a tyrosine kinase inhibitor e.g., sorafenib.

26. The method of any one of embodiments 1 to 25, wherein Compound 1 or a pharmaceutically acceptable salt is orally administered to the patient once or twice daily.

27. The method of embodiment 26, wherein 100 mg to 300 mg of Compound 1 or an equivalent amount of a pharmaceutically acceptable salt of Compound 1 is administered twice daily.

28. The method of embodiment 27, wherein 100 mg, 150 mg, 200 mg, or 300 mg of Compound 1 or an equivalent amount of a pharmaceutically acceptable salt of Compound 1 is administered twice daily.

29. The method of embodiment 28, wherein 100 mg of Compound 1 or an equivalent amount of a pharmaceutically acceptable salt of Compound 1 is administered twice daily.

30. The method of embodiment 26, wherein the total daily dose of Compound 1 or an equivalent amount of a pharmaceutically acceptable salt of Compound 1 is 600 mg or less.

31. The method of embodiment 26, wherein the total daily dose of Compound 1 or an equivalent amount of a pharmaceutically acceptable salt of Compound 1 is 200 mg.

32. The method of embodiment 26, wherein the total daily dose of Compound 1 or an equivalent amount of a pharmaceutically acceptable salt of Compound 1 is 300 mg.

33. The method of embodiment 26, wherein the total daily dose of Compound 1 or an equivalent amount of a pharmaceutically acceptable salt of Compound 1 is 400 mg.

34. The method of embodiment 26, wherein Compound 1 or a pharmaceutically acceptable salt thereof is administered once in the morning and once in the evening.

35. The method of embodiment 34, wherein the time between administrations is ten to fourteen hours.

36. The method of embodiment 34 or 35, wherein the time between administrations is at least eight hours.

37. The method of any one of embodiments 26 to 36, wherein Compound 1 or a pharmaceutically acceptable salt thereof is administered twice daily and the patient experiences at least one side effect reduction.

38. The method of embodiment 37, wherein the at least one side effect reduction is chosen from reduced diarrhea, reduced nausea, reduced vomiting, reduced ALT increase, reduced AST increase, and reduced abdominal pain.

39. The method of any one of embodiments 26 to 36, wherein Compound 1 or a pharmaceutically acceptable salt thereof is administered twice daily and the patient experiences at least one improved clinical outcome.

40. The method of embodiment 39, wherein the at least one improved clinical outcome is chosen from improved median time to progression (TTP), improved three- and six-month progression-free survival (PFS), and improved overall survival (OS).

41. The method of any one of embodiments 1 to 41, wherein 50 mg to 150 mg of palbociclib is orally administered to the patient once daily.

42. The method of embodiment 41, wherein the total daily dose of palbociclib is 125 mg.

43. The method of embodiment 41, wherein the total daily dose of palbociclib is less than 125 mg.

44. The method of any one of embodiments 41 to 43, wherein palbociclib is administered to the patient with food.

45. The method of any one of embodiments 41 to 44, wherein palbociclib is administered to the patient for twenty-one consecutive days, followed by seven days in which no palbociclib is administered to the patient.

46. The method of embodiment 45, wherein the twenty-eight day administration schedule is repeated one or more times.

47. A combination therapy comprising at least one selective fibroblast growth factor receptor 4 (FGFR4) inhibitor and at least one cyclin-dependent kinase 4/6 (CDK4/6) inhibitor.

48. The combination therapy of embodiment 47 or 48, wherein the at least one selective FGFR4 inhibitor is chosen from N-((3S,4S)-3-((6-(2,6-dichloro-3,5-dimethoxyphenyl)quinazolin-2-yl)amino)tetrahydro-2H-pyran-4-yl)acrylamide (Compound 1), N-(2-((6-(2,6-dichloro-3,5-dimethoxyphenyl)quinazolin-2-yl)amino)-3-methylphenyl)acrylamide (Compound 2), and pharmaceutically acceptable salts thereof.

49. The combination therapy of any one of embodiments 47 to 49, wherein the at least one CDK inhibitor is chosen from palbociclib and pharmaceutically acceptable salts thereof.

50. A method of treating a cancer in a patient in need thereof comprising:
a. determining if, having determined if, or receiving information that the patient has a cancer characterized by at least one biomarker chosen from fibroblast growth factor 19 (FGF19) overexpression, amplified FGF19, and fibroblast growth factor receptor 4 (FGFR4) overexpression;
b. identifying the patient as responsive to a combination therapy of any one of embodiments 47 to 49; and
c. administering a therapeutically effective amount of the combination therapy to the patient.

51. A method of treating a cancer in a patient in need thereof comprising administering a therapeutically effective amount of a combination therapy of any one of embodiments 47 to 49 to a patient having a cancer characterized by at least one biomarker chosen from fibroblast growth factor 19 (FGF19) overexpression, amplified FGF19, and fibroblast growth factor receptor 4 (FGFR4) overexpression, wherein the cancer is responsive to the combination therapy.

### EXAMPLES

The following examples are intended to be illustrative and are not meant to limit the scope of the disclosure.

### Example 1

### Palbociclib and Compound 1 Combination Studies in Cells

Combinations of FGFR4 and CDK4/6 inhibitors were evaluated in several signal seeking cell-based in vitro assays (data not shown). Screening was carried out using a variety of different standard anti-proliferative assays such as e.g., MTS, MTT, and Cell Titer Glo®. Many of the cell lines tested showed sensitivity, including in some instances partial response e.g., ZR-75-1, SW1116, TE-8, SNU-761, SNU-878, or in some instances synergistic response e.g., JHH7, MDA-MB-453, Huh-7. Not all cell lines showed sensitivity, which may be due to a variety of reasons. For example, a lack of sensitivity was observed in cell lines that were resistant to either agent alone, such as cell lines not having an intact FGFR4 signaling pathway e.g., JHH4. In other cell lines, limitations related to the in vitro assay format or time point of the readout may impact activity. As has been reported previously, palbociclib activity can be weak in short-term assays or in assays using general readouts (such as metabolic intermediates) not specific for cell proliferation (Gao et al., Nature Medicine, 2015, 21(11), 1318-1325). However, taken together, based on positive signals observed in the cell-based in vitro assays, mechanism of action and in vivo PDX studies were pursued.

Potential synergistic interactions between palbociclib and Compound 1 were assessed in Huh-7 cells (e.g. Riken RCB, #RCB1366) relative to the Loewe additivity model using CHALICE™ software (Horizon CombinatoRx Inc., Cambridge, Mass.), via a synergy score calculated from the difference between the observed values and those predicted from the Loewe additivity model across a range of concentrations of each compound. A five point dilution series of palbociclib (10 nM, 30 nM, 100 nM, 300 nM, 1 μM) was combined with an eight point dilution series of Compound 1 with a dose range 0.11 nM to 0.25 μM (0.11 nM, 0.34 nM, 1.03 nM, 3.1 nM, 9.25 nM, 27.8 nM, 83.3 nM, and 250 nM) to create a drug combination matrix that spanned 45 distinct combinations. Specifically, the study was conducted according to the following protocol: On Day 1, Huh-7 cells were trypsinized, centrifuged, and counted on the cell counter. Cells were plated in a clear, flat-bottom tissue culture treated 96-well plate at 3000 cells/well. The cells were placed in 37° C. incubator overnight to allow cells to attach. On Day 2, two mother compound plates were made up with serial dilutions of either Compound 1 (250nM starting concentration) or palbociclib (luM starting concentration). Mother plates were combined into a single daughter plate (mixing 1:1 the compounds from each mother plate). Using the Bravo liquid handler, the duplicate cell plates were dosed with compounds from the daughter plate. Cells were allowed to grow in the incubator for 3 days. On Day 5, after 3 days, the plates were developed. One plate was developed for BrdU per the BrdU kit protocol from Sigma. A Second plate was developed using Cell Titer-Glo. The following materials were utilized in the above studies: DMEM (ThermoFisher Scientific, #11965092), Fetal bovine serum (Gemini), Pen/strep (ThermoFisher Scientific, #15140163), Cell Proliferation ELISA, BrdU colorimetric (Sigma, #11647229001), CellTiter-Glo Luminescent Cell Viability Assay (Promega, #G7570). Data Analysis was performed as described below: BrdU proliferation data analyzed using Chalice Analyzer (Horizon Discovery) as described below. CellTiter-Glo data analyzed using Excel (Microsoft).

Relative proliferation was measured using the CellTiter-Glo (CTG) assay (Promega). Percent inhibition of cell proliferation values were calculated as:

$$1 - \frac{\begin{matrix}[ ( Average Combination Well Signal ) - \\ ( Average Staurosporine Signal ) ]\end{matrix}}{\begin{matrix}[ ( Average DMSO Signal ) - \\ ( Average Staurosporine Signal )\end{matrix}}$$

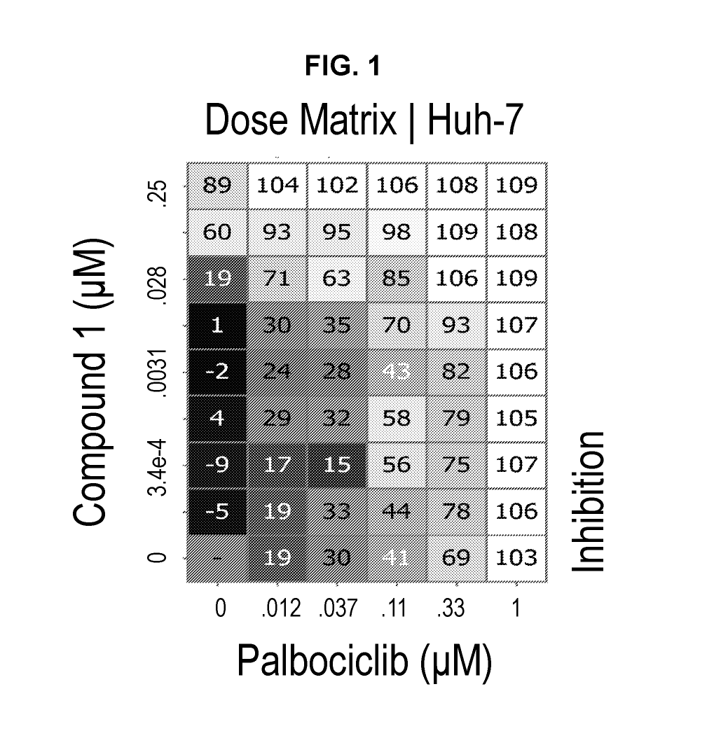

and entered into the CHALICE software to generate a synergy score and an isobologram to visualize any excess inhibition or potency shifts obtained with compounds tested in combination. Synergistic growth inhibition occurred when the combined effect of two compounds was greater than what was predicted based on the Loewe additivity model for the compound combination. This method of assessing synergistic growth inhibition is explained in detail in Lehar et al. (Nat. Biotechnology, 2009, 27, 659-666) and the CHALICE software technical guide. A table showing synergistic growth inhibition in cells for the combination of Compound 1 and palbociclib is shown in FIG. 1.

### Example 2

### Growth Inhibition in Cells Treated with Palbociclib and/or Compound 1

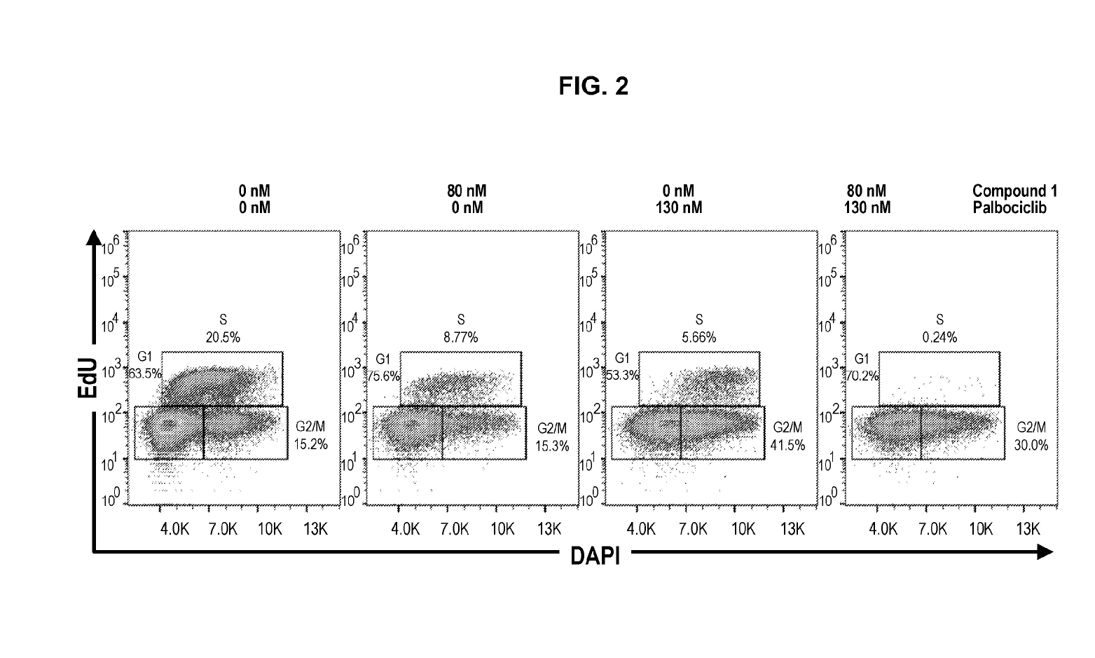

Huh-7 cells were treated with the indicated concentrations of Compound 1, palbociclib, or a combination of Compound 1 and palbociclib for 72 hours. Following compound incubation, Edu was added to the culture medium at a final concentration of 10 μM for 2 hours. Cells were then harvested and washed with 1% BSA in PBS, pelleted, and resuspended in 100 μL of Click-iT™ fixative (Invitrogen, Click-iT™ Plus EdU Flow Cytometry Assay Kit). Cells were incubated with the fixative for 15 minutes at room temperature, protected from light. Next, the cells were washed as described previously, and resuspended in 100 μL of 1× Click-iT™ saponin-based permeabilization and wash reagent and incubated with the reagent for 15 minutes at room temperature, protected from light. 500 μL of Click-iT™ Plus reaction cocktail was then added and cells were incubated for 30 minutes at room temperature, protected from light. Next, cells were washed with 3 mL of 1× Click-iTTM saponin-based permeabilization and wash reagent and resuspended in 500 μL of the same permeabilization and wash reagent. 5 μL of a 50 μM DAPI solution was then added to the cells and total DNA content was analyzed on the Attune™ Flow Cytometer with the appropriate filters. No cell division was observed in cells treated with both 80 nM Compound 1 and 130 nM palbociclib. Cell cycle analysis showed the majority of cells were trapped in the G1 phase of the cell cycle following treatment with the combination, as shown in FIG. 2.

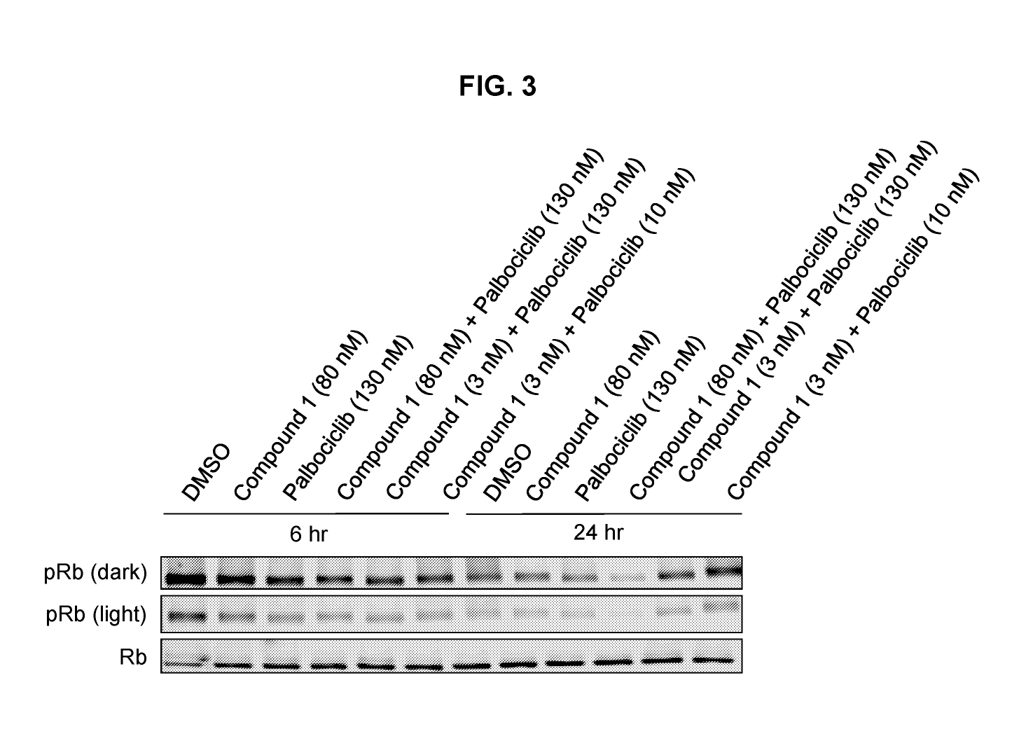

As shown in FIG. 3, after treatment of Huh-7 cells for the indicated times, cells were stimulated with 100 ng/mL of FGF19 for 10 minutes. Cells were then pelleted and lysed in cell extraction buffer (Life Technologies), containing 1× protease and 1× phosphatase inhibitor cocktail (Sigma). Total protein concentration was determined using a standard Bradford assay. Western blotting was performed on cell lysates normalized to 50 μg/total protein in loading buffer (Life Technologies). Normalized lysates were run on SDS-PAGE and transferred to a nitrocellulose membrane (Life Technologies). The membrane was incubated overnight at 4° C. with primary antibodies (1:1,000). Antibodies used in these studies were from Cell Signaling Technologies [anti-phospho-RB (S807/811)] and Abcam [anti-RB (Rb1 1F8) ab24]. Membranes were washed, incubated with IRDye secondary antibodies (LI-COR), washed again, and imaged on an Odyssey Fc (LI-COR) (FIG. 3).

### Example 3

### Palbociclib and Compound 1 Combination Studies in Female Balb/c Nude Mice Bearing Xenografts

Female Balb/c nude mice (Mus Musculus) between six and eight weeks old and weighing 18 to 20 g were used to evaluate the therapeutic efficacy of palbociclib and Compound 1 as monotherapies and in combination in Huh-7 liver cancer xenograft models. The tumor cells were maintained in vitro as a monolayer culture in DMEM medium supplemented with 10% heat inactivated fetal bovine serum at 37° C. in a 5% CO₂ atmosphere. The tumor cells were routinely subcultured twice weekly by trypsin-EDTA treatment. The cells growing in an exponential growth phase were harvested and counted for tumor inoculation.

Each mouse was inoculated subcutaneously at the right flank with tumor cells (5×10⁶) in 0.2 ml of PBS supplemented with Matrigel (50:50) for tumor development. The treatments were started on day 11 after tumor inoculation when the average tumor size reached approximately 183 mm³. Each group consisted of nine tumor-bearing mice.

Tumor size was measured twice weekly in two dimensions using a caliper, and the volume was expressed in mm³ using the formula:

V=0.5a×b²

where a and b are the long and short diameters of the tumor, respectively. The tumor size was then used to calculate TGI and T/C values.

TGI was calculated for each group using the formula:

$$TGI ( \% ) = \frac{1 - ( Ti - T 0 )}{Vi - Vo} \times 100$$

where Ti is the average tumor volume of a treatment group on a given day; T0 is the average tumor volume of the treatment group on the day treatment began; Vi is the average tumor volume of the vehicle control group on the same day Ti was measured, and V0 is the average tumor volume of the vehicle group on the day treatment began. The T/C value (in percent) is an indication of antitumor effectiveness. T and C are the mean volumes of the treated and control groups, respectively, on a given day.

Summary statistics, including mean and the standard error of the mean (SEM), were provided for the tumor volume of each group at each time point. For tumor volume comparison among more than two groups, a one-way ANOVA was performed. For comparisons between two groups, a t-test was performed. All analysis was run in GraphPad Prism 5.0, with p<0.05 considered to be statistically significant.

For all groups, at four hours after last dosing, tumor samples were collected in less than one minute after the euthanasia. Four tumors in each group were snap frozen; four tumors were fixed and made into FFPE.

The average tumor size of vehicle-treated animals reached 2824 mm³ on day eighteen. All single treatment groups and all combination treatment groups showed statistically significant difference in antitumor efficacy when compared with the vehicle group.

Vehicle group, Compound 1 30 mg/kg group and palbociclib 45 mg/kg groups were taken down on day eighteen because the average tumor volume reached 2000 mm³. Other groups were taken down on day twenty-one. Treatment with Compound 1 (30 mg/kg bid), Compound 1 (100 mg/kg bid), and Compound 1 (200 mg/kg bid) showed statistically significant and dose-dependent antitumor activity when compared with the vehicle group; average tumor size were measured to be 1612 mm³ (T/C value=57%; p<0.001), 690 mm³ (T/C value=24%; p<0.001) and 198 mm³ (T/C value=7%; p<0.001), respectively, on day eighteen. Treatment with palbociclib at 45 mg/kg and 90 mg/kg also showed statistically significant and dose-dependent antitumor activity when compared with the vehicle group; average tumor size was measured to be 1992 mm³ (T/C value=71%; p<0.001) and 1169 mm³ (T/C value=41%; p<0.001), respectively, on day eighteen.

Combination treatment with Compound 1 (30 mg/kg) and palbociclib (45 mg/kg), Compound 1 (100 mg/kg) and palbociclib (45 mg/kg), Compound 1 (200 mg/kg) and palbociclib (45 mg/kg), and Compound 1 (30 mg/kg) and palbociclib (90 mg/kg) showed statistically significant antitumor efficacy when compared with the vehicle group; average tumor size were measured to be 630 mm³ (T/C value=22%; p<0.001), 254 mm³ (T/C value=9%; p<0.001), 230 mm³ (T/C value=8%; p<0.001), and 247 mm³ (T/C value=9%; p<0.001), respectively, on day eighteen.

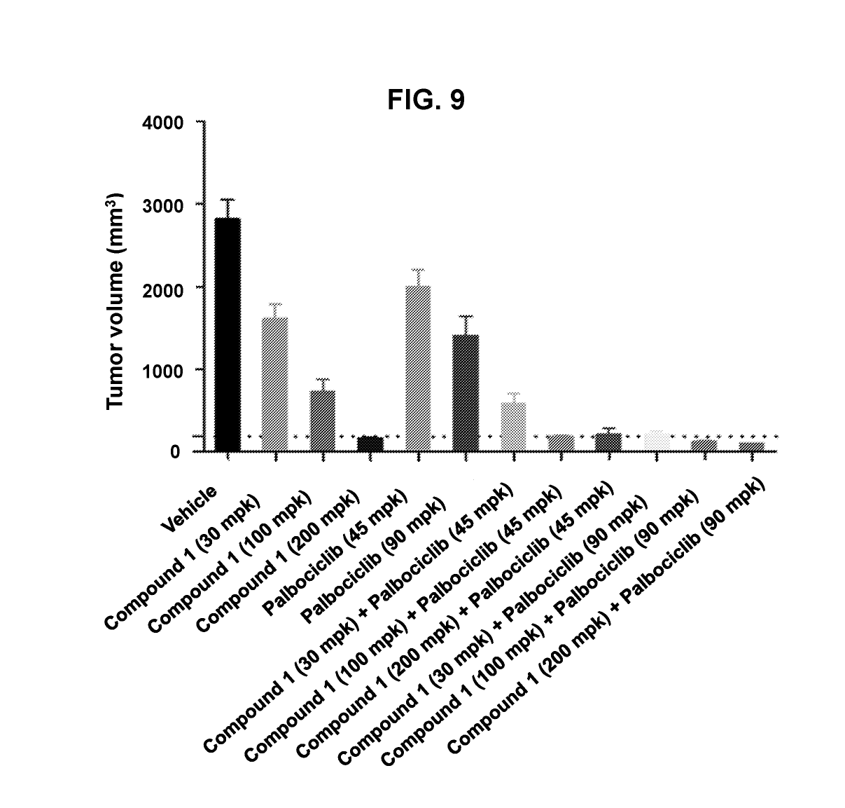

Combination treatment with Compound 1 (100 mg/kg) and palbociclib (90 mg/kg) and Compound 1 (200 mg/kg) and palbociclib (90 mg/kg) caused tumor regression; average tumor size were measured to be 164 mm³ (T/C value=6%; p<0.001) and 122 mm³ (T/C value=4%; p<0.001), respectively, on day eighteen. These results are summarized in FIG. 9.

When compared with Compound 1 (30 mg/kg) and Compound 1 (100 mg/kg) monotherapies, combinations of Compound 1 with palbociclib showed significantly improved antitumor efficacy.

Similarly, the combination of palbociclib (90 mg/kg) with Compound 1 (200 mg/kg) showed significantly improved antitumor efficacy relative to the Compound 1 (200 mg/kg) monotherapy.

One animal in the palbociclib (90 mg/kg) group lost 15.5% body weight; its dosing was stopped on day seven. The animal was euthanized on day fourteen due to >20% body weight loss. One mouse in the Compound 1 (200 mg/kg) and palbociclib (45 mg/kg) group lost 15.6% body weight; its dosing was stopped since day five and resumed since day fourteen. Palbociclib treated animals were supplied with sunflower seeds to mitigate body weight loss on and after day eight per the sponsor's approval. All other animals maintained their body weight.

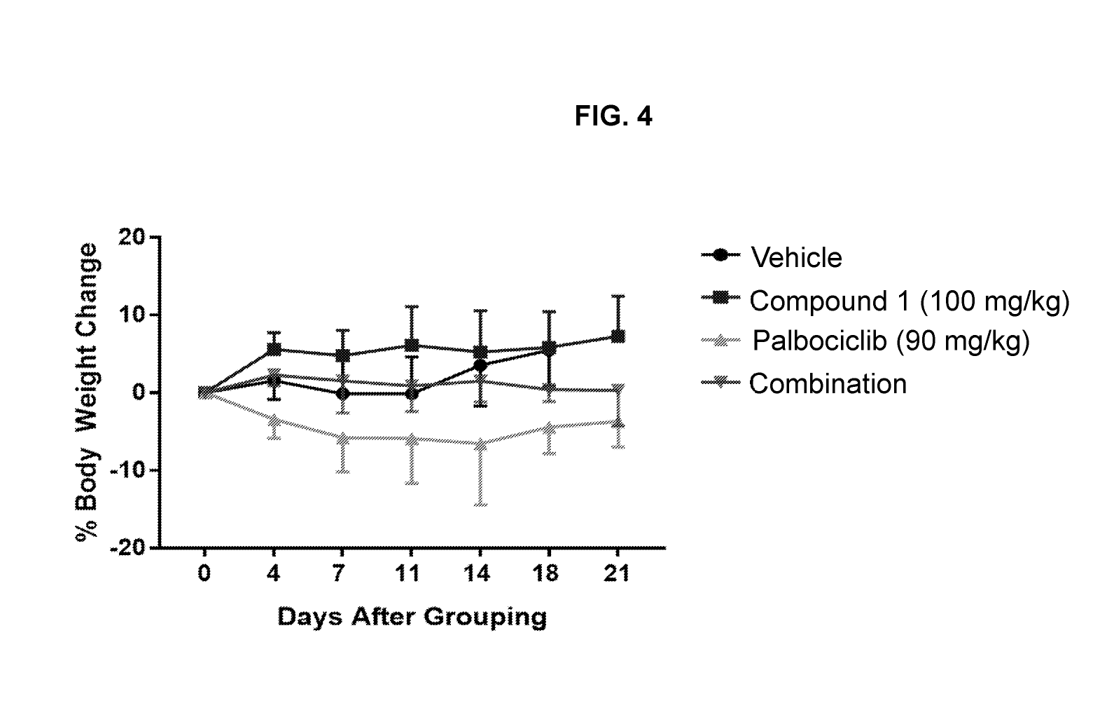

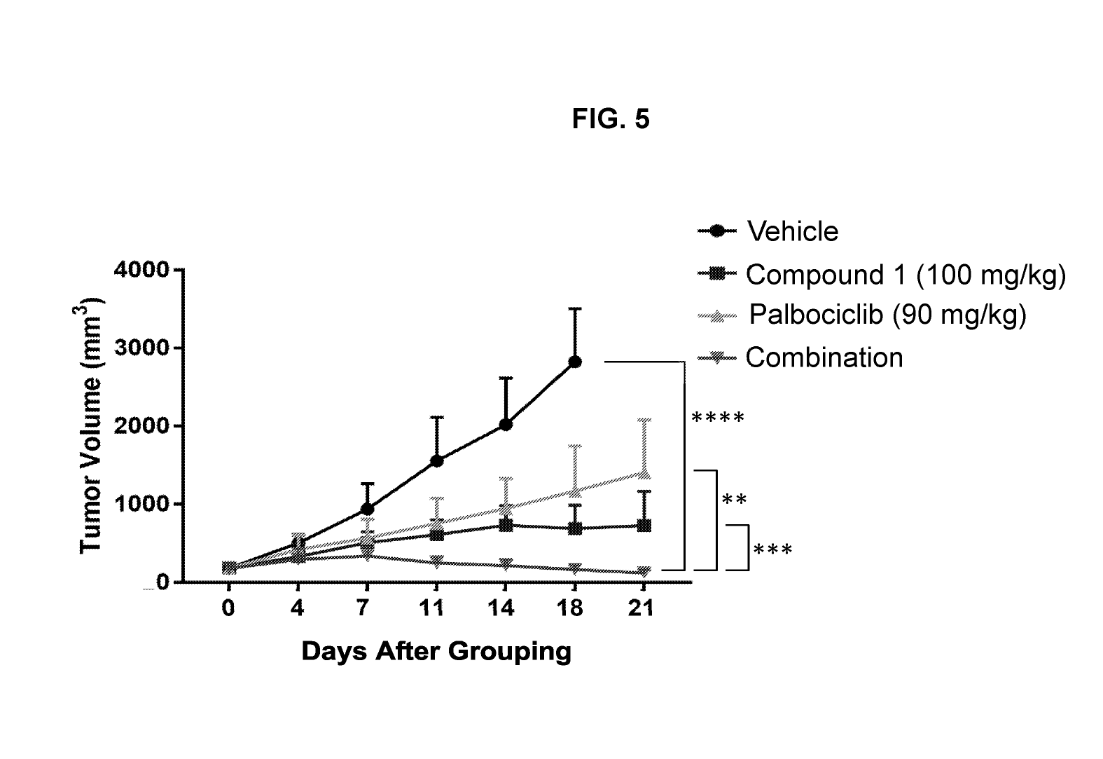

The percentage body weight change for female Balb/c nude mice bearing xenografts treated with vehicle, Compound 1 (100 mg/kg), palbociclib (90 mg/kg), or the combination of Compound 1 (100 mg/kg) and palbociclib (90 mg/kg) is shown in FIG. 4. Data are shown as mean±SEM. FIG. 5 shows tumor volume traces for female Balb/c nude mice bearing xenografts treated with vehicle, Compound 1 (100 mg/kg), palbociclib (90 mg/kg), or the combination of Compound 1 (100 mg/kg) and palbociclib (90 mg/kg). Data are shown as mean±SEM.

### Example 4

### Immunohistochemical Studies in Xenograft Tumors

A study was performed to evaluate H&E staining and immunohistochemical positivity (Ki-67 and p Histone H3) in tumor samples at the end of the study described in Example 3. Tumor tissue blocks were sectioned at 5 μm thickness and mounted on charged slides. Slides were deparaffinized, rehydrated and either routine H&E stained or IHC stained for presence of Ki67 (e.g., Abcam, ab16667 Clone [SP6], 1:250 dilution) or phospho-Histone H3 (e.g., Millipore, 04-1093 Clone E173, 1:1000 dilution) on the Leica Bond staining platform with HistoTox standard ER1 30 minute rabbit protocol. IHC slides were counterstained with hematoxylin offline, dehydrated, and permanently coverslipped.

Samples were processed routinely, sectioned at approximately 5 microns, and were either H&E stained, or IHC stained for Ki-67 or p-Histone H3. IHC glass slides were scanned (at 20×) using an Aperio AT2 whole slide scanner. Image analysis was performed on the digital slide images using Visiopharm software. Image analysis utilized the following procedures:

Tumor sections were identified using an automated algorithm to detect tissue. Tumors were outlined as a Region of Interest (ROI). Manual alterations were performed to ensure accurate ROIs.

The tissue ROIs were processed using imaging filters in order to separate positive staining from counterstaining. Imaging filters involve color deconvolution methods relating to the image's pixel values.

Processed images were classified using a thresholding method, where a threshold is established based on pixel values associated with positively stained tissue (Ki-67 or p-Histone H3). This separated the different tissue types by applying a label to the positive tissue.

For p-Histone H3, only positive nuclei present in the correct tissue type were counted as positive. Quantification of the amount of positive tissue was determined by analyzing the labeled image.

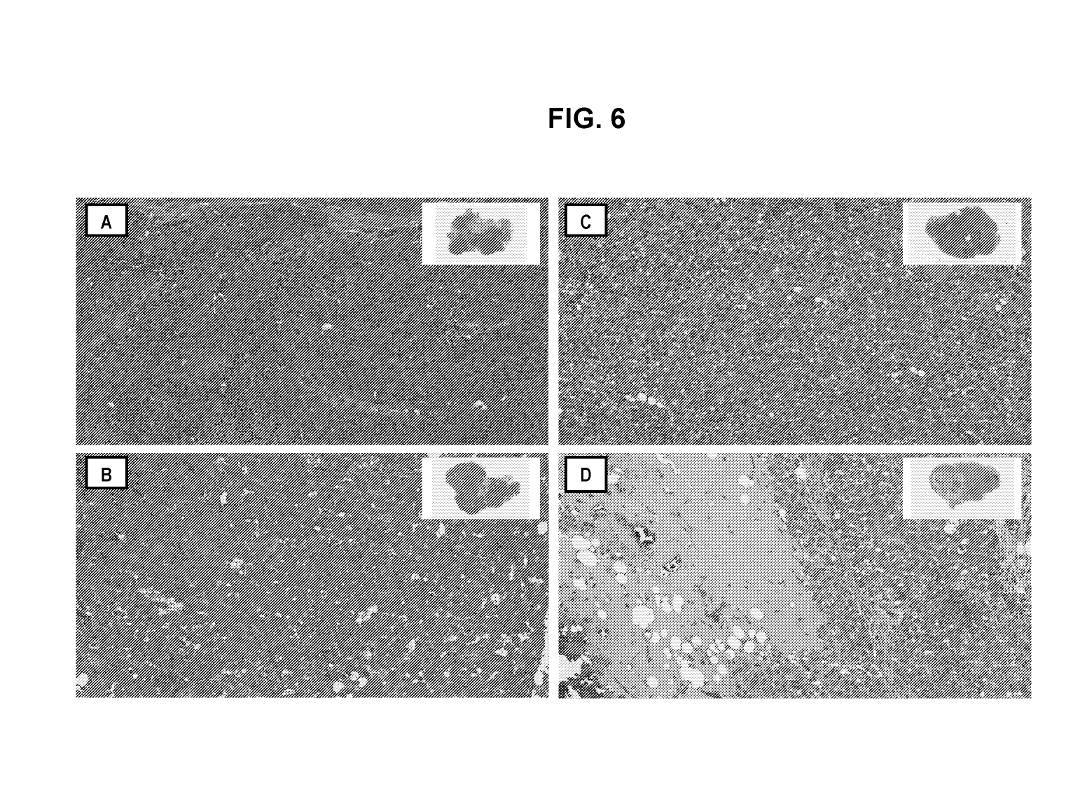

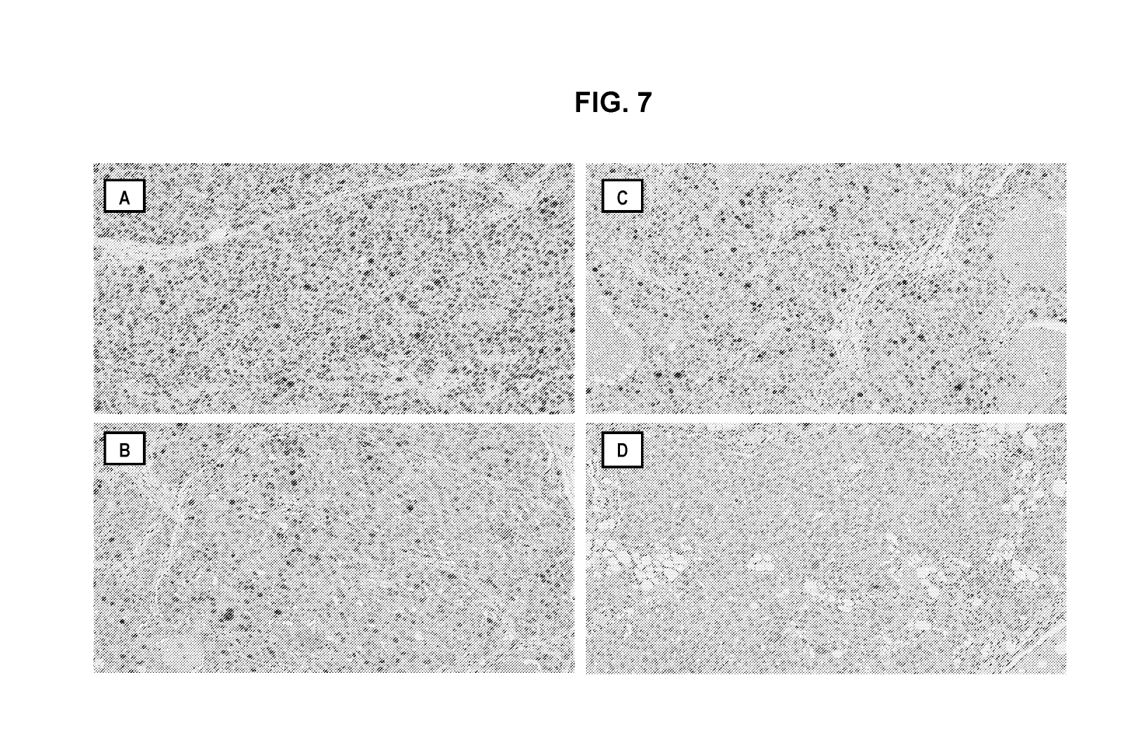

For Ki-67, parameters associated with the area of the tumor, number of positive cells, total number of cells, and nuclear density were output as raw data. Ki-67 nuclear density was calculated as number of Ki-67 positive nuclei divided by the total area multiplied by 1000000 for mm². Photomicrographs are shown in FIG. 6 of H&E staining and in FIG. 7 of Ki67 staining of xenograft tumors from Balb/c nude FGF-19 amplified Huh-7 mice treated with vehicle (A), palbociclib (B), Compound 1 (C), or the combination of Compound 1 and palbociclib (D). Immunohistochemistry demonstrated greater inhibition of the expression of proliferation marker Ki67 after combination treatment over single agent.

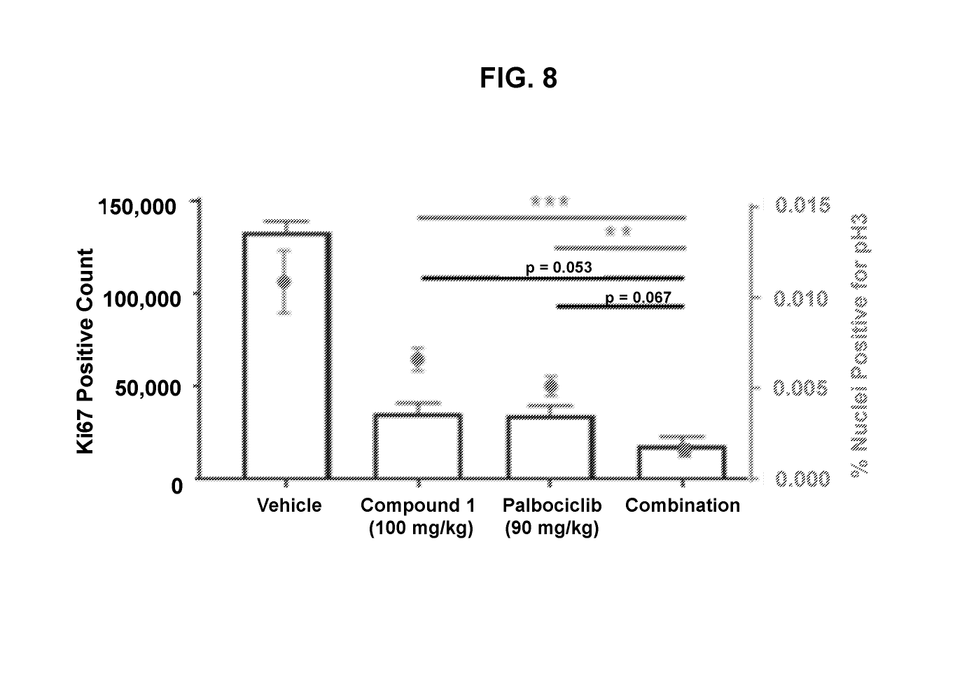

For p-Histone H3, parameters associated with the number of positive cells, number of negative cells, total number of cells, and percent of positive p-Histone H3 cells were output as raw data (data not shown). Significant inhibition of the expression of proliferation marker p-Histone H3 was also seen by immunohistochemistry after combination treatment over single agent (FIG. 8).

### Example 5

### Compound 1 Studies with Hep3B and LIX-066 Tumor Models In Vivo Efficacy Study of Compound 1 in LIX-066 Xenograft Model

LIX-066 was a human primary hepatocellular carcinoma model from ChemPartner. 130 mice were implanted for this study. Two animals were designated as sentinels; 2 animals were designated as extras.

Animals were monitored daily by general clinical observation throughout the study period. Body weights were recorded twice weekly before randomization and recorded every day during dosing period and the dose was adjusted per body weight. Tumor areas (length×width) was measured two to three times per week by using digimatic callipers throughout the study period and tumor volumes were calculated based on the following formula: tumor volume=(length×width)/2. The tumor volume was then used for calculations of net tumor growth inhibition (TGIn) values. The TGIn (in percent) is the indication of anti-tumor activity. TGIn (%)=[1−avTi-₀/avCi-₀)]×100; avTi-₀ is the average of the tumor volume of each mouse in the treatment group on a specific day minus the tumor volume of each mouse in the treatment group on the first day; avCi-₀ is the average of the tumor volume of each mouse in the Vehicle control group on a specific day minus the tumor volume of each mouse in the Vehicle control group on the first day of treatment. Efficacy data was graphically represented as the mean tumor volume±standard error of the mean (SEM).

Based on about 40% take rate, 50 animals were selected for efficacy study when their tumors reach appropriate size (100-300 mm³). Five animals were used for plasma PK standard control. All other animals were euthanized. The 50 animals were randomized (blocked randomization/using Excel software) and divided into the following groups for compound administration:

Group 1: N=10; Vehicle control (PEG400: 20% HP-γ-CD=3:2)

Group 2: N=10; Compound 1, 30 mg/kg, bid, po

Group 3: N=10; Compound 1, 100 mg/kg, bid, po

Group 4: N=10; Compound 1, 200 mg/kg, bid, po

All animals were monitored daily for clinical observation (animal mortality, appearance, spontaneous activity, body posture, and food and water intake. Any lesions and adverse reactions were recorded). Any animals showing signs of debilitation, marked body weight loss (>20%), cachexia or large tumors that would inhibit an animal's ability to eat and drink or mobility were euthanized immediately. Any animals with severely ulcerated, infected or severely hemorrhagic tumors, or tumors whose estimated weight exceed 20% of the body weight were euthanized.

The mice were euthanized and the tumor weights were measured without sampling.

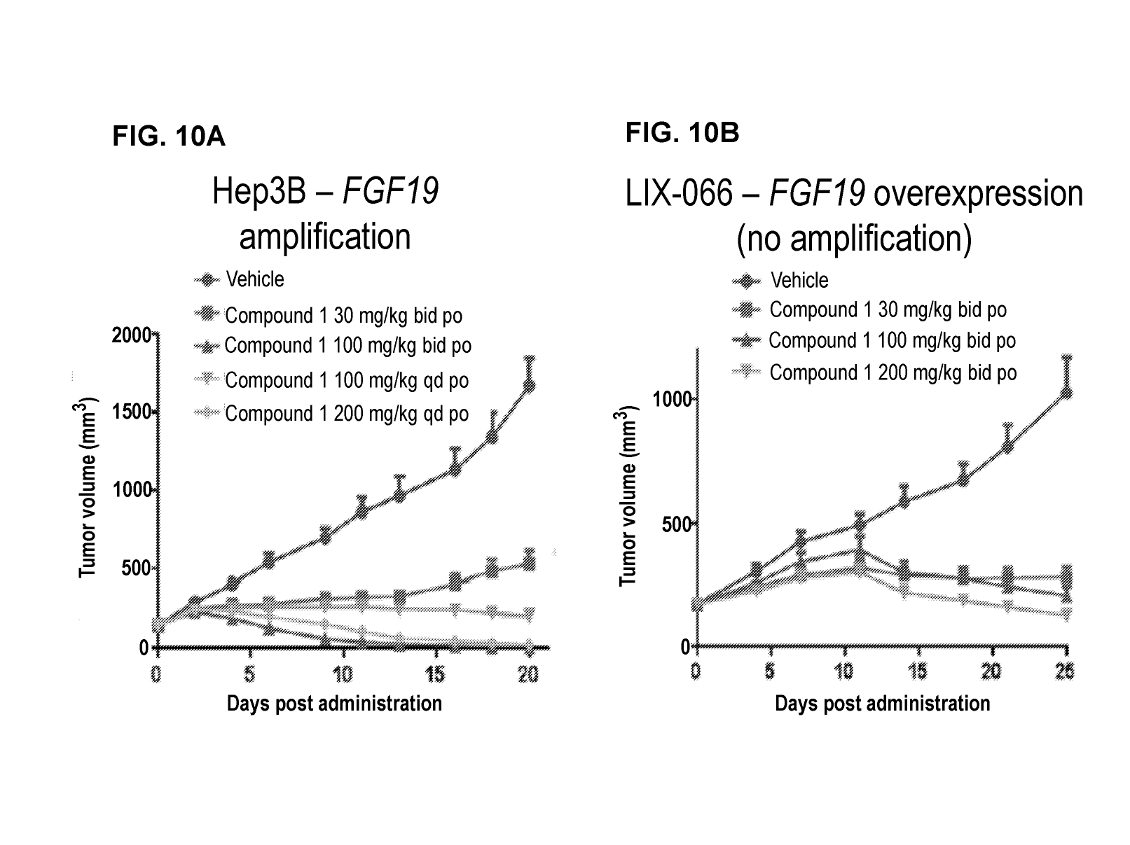

The tumor growth curves during administration of each group are presented in FIG. 10B. The average tumor volumes of the 5 groups at D28 were 1278.72 mm³, 262.87 mm³, 168.03 mm³, and 112.22 mm³, respectively. Compound 1 at all levels (30 mg/kg BID,100 mg/kg BID and 200 mg/kg BID) significantly inhibits tumor growth of LIX066 model withp<0.01 compared with Vehicle group.

An inital study (dose and model not optimized) for evaluating Compound 1 in combination with palbociclib in an HCC patient-derived xenograft model LIX066 showed that cotreatment with palbociclib did not further enhance anti-tumor efficacy.

A similar study as described above was carried out using Hep3B-FGF19 amplified mice. Tumor volume results are shown in (FIG. 10A). Compound 1 monotherapy induced Hep3B tumor shrinkage (100% regression, >20% CRs). Compound 1 was well tolerated in both of these tumor models.

### Example 6

### Prophetic Combination Study in Mice

The objective of this study is (1) to identify mouse models with liver cancer characterized by FGF19 expression, but with no detectable FGF19 amplification, wild type FGFR4 wild type R^{B}, wild type KLB, and that show pathway inhibition upon combination treatment. Pathway inhibition is measured by decreases in phosphorylated R^{B} and (2) once certain models are identified that show an appropriate dose response, the selective models are utilized for an efficacy study (Vehicle, Compound 1 only, palbociclib only, combination of Compound 1 and palbociclib).

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the disclosure described herein. Such equivalents are intended to be encompassed by the following claims.

1. A method for treating a cancer characterized by amplified FGF19 in a patient in need thereof comprising administering a therapeutically effective amount of at least one fibroblast growth factor receptor 4 (FGFR4) inhibitor in combination with at least one cyclin-dependent kinase (CDK) inhibitor to the patient, wherein:
the at least one CDK inhibitor is chosen from CDK4/6 inhibitors; and
the at least one FGFR4 inhibitor is chosen from N-((3S,4S)-3-((6-(2,6-dichloro-3,5-dimethoxyphenyl)quinazolin-2-yl)amino)tetrahydro-2H-pyran-4-yl)acrylamide (Compound 1), N-(2-((6-(2,6-dichloro-3,5-dimethoxyphenyl)quinazolin-2-yl)amino)-3-methylphenyl)acrylamide (Compound 2), and pharmaceutically acceptable salts thereof.

2. (canceled)

3. The method of claim 1, wherein the at least one CDK inhibitor is chosen from palbociclib, 7-cyclopentyl-N,N-dimethyl-2-((5-(piperazin-1-yl)pyridin-2-yl)amino)-7H-pyrrolo[2,3-d]pyrimidine-6-carboxamide, 2-(2-chlorophenyl)-5,7-dihydroxy-8-[(3S,4R)-3-hydroxy-1-methyl-4-piperidinyl]-4-chromenone, N-(5-((4-ethylpiperazin-1-yl)methyl)pyridin-2-yl)-5-fluoro-4-(4-fluoro-1-isopropyl-2-methyl-1H-benzo[d]imidazol-6-yl)pyrimidin-2-amine, GZ38-1, and pharmaceutically acceptable salts thereof.

4. The method of claim 1, wherein the at least one CDK inhibitor is chosen from palbociclib and pharmaceutically acceptable salts thereof.

5-7. (canceled)

8. The method of claim 1, wherein the cancer is further characterized by fibroblast growth factor 19 (FGF19) overexpression.

9. (canceled)

10. The method of claim 8, wherein the cancer is further characterized by wild-type retinoblastoma protein and wild type klotho beta.

11-13. (canceled)

14. The method of claim 1, wherein the cancer is further characterized by FGFR4 overexpression.

15. The method of claim 1, wherein the cancer is chosen from breast cancer, ovarian cancer, lung cancer, liver cancer, a sarcoma, esophagus cancer, large intestine cancer, colon cancer, head and neck cancer, and hyperlipidemia.

16. The method of claim 15, wherein the cancer is liver cancer.

17. The method of claim 16, wherein the cancer is hepatocellular carcinoma or hepatoblastoma.

18. The method of claim 17, wherein the cancer is fibrolamellar hepatocellular carcinoma.

19. The method of claim 17, wherein the cancer is unresectable hepatocellular carcinoma.

20. The method of claim 15, wherein the cancer is breast cancer.

21. The method of claim 20, wherein the cancer is metastatic breast cancer.

22. The method of claim 21, wherein the cancer is receptor (ER)-positive, human epidermal growth factor receptor 2 (HER2)-negative metastatic breast cancer.

23. The method of claim 1, wherein the patient is a human.

24. (canceled)

25. (canceled)

26. The method of claim 1, wherein Compound 1 or a pharmaceutically acceptable salt is orally administered to the patient once or twice daily.

27. The method of claim 26, wherein 100 mg to 300 mg of Compound 1 or an equivalent amount of a pharmaceutically acceptable salt of Compound 1 is administered twice daily.

28-40. (canceled)

41. The method of claim 4, wherein 50 mg to 150 mg of palbociclib is orally administered to the patient once daily.

42-44. (canceled)

45. The method of claim 4, wherein palbociclib is administered to the patient for twenty-one consecutive days, followed by seven days in which no palbociclib is administered to the patient.

46. The method of claim 45, wherein the twenty-eight day administration schedule is repeated one or more times.

47-51. (canceled)
